(19) United States
(12) Patent Application Publication
(10) Pub. No.: US 2010/0222432 A1
(43) Pub. Date: Sep. 2, 2010

(54) Synthetic Carbon Nanotubes

(75) Inventor: Duy H. Hua, Manhattan, KS (US)

Correspondence Address: GREENLEE WINNER AND SULLIVAN P C, 4875 PEARL EAST CIRCLE, SUITE 200, BOULDER, CO, 80301, US

(73) Assignee: Kansas State University Research Foundation

(21) Appl. No.: 12/063,101

(22) PCT Filed: Aug. 10, 2006
(86) PCT No.: PCT/US06/31322

§ 371 Date: Sep. 2, 2008

Related U.S. Application Data
(60) Provisional application No. 60/707,256, filed on Aug. 11, 2005.

(51) Int. Cl. A61K 31/165; A61K 9/14; A61P 1/18; A61P 15/08; D01F 9/12; C07C 237/48; A61P 9/00

(52) U.S. Cl. 514/616; 424/489; 428/367; 423/447.1; 423/447.2; 564/153; 977/906; 977/742

(57) ABSTRACT

Methods to prepare synthetic carbon nanotubes having controllable properties and synthetic carbon nanotubes having controllable properties are provided. The properties which are controllable using the methods provided here include independently and in combination: diameter, length, identity and number of functional groups present and identity and number of heteroatoms present.

### CROSS-REFERENCE TO RELATED APPLICATIONS

This application claims priority to U.S. provisional application Ser. No. 60/707,256, filed Aug. 11, 2005, which is incorporated by reference herein to the extent not inconsistent with the disclosure herewith.

### STATEMENT REGARDING FEDERALLY SPONSORED RESEARCH OR DEVELOPMENT

This invention was made with U.S. government support under grant number CHE-0555341 awarded by the National Science Foundation. The U.S. government has certain rights in the invention.

### BACKGROUND OF THE INVENTION

Carbon nanotubes are allotropes of carbon comprising one or more cylindrically configured graphene sheets and are classified on the basis of structure as either single walled carbon nanotubes (SWNTs) or multiwalled carbon nanotubes (MWNTs). SWNTs consist of a single graphite sheet wrapped into a cylindrical tube, and MWNTs are an array of many SWNTs that are concentrically formed like rings of a tree trunk. Typically having small diameters (≈1-30 nanometers) and large lengths (up to several microns), SWNTs and MWNTs commonly exhibit very large aspect ratios (i.e., length to diameter ratio 10³ to about 10⁵). Shorter nanotubes are preferable for further chemical manipulation.

Currently, MWNTs and SWNTs are made from high-pressure CO conversion, pulsed-laser vaporization, chemical vapor deposition, or carbon arc synthesis. When the length of SWNTs increases, the solubility decreases. Nanotubes generated using these methods are insoluble in organic solvents. Derivatization of SWNTs is required to enhance the solubility in organic solvents. Derivatization processes currently in use produce materials with a random arrangement of chemical modifications.

SWNTs with diameter distributions peaked at ˜0.9 and 1.3 nm have been reported, but larger diameter SWNTs (>1.5 nm) have not been reported. MWNTs typically have outer diameters ranging from 2.5 nm to 30 nm. Both SWNTs and MWNTs are closed at both ends with caps, which contain pentagonal carbon rings. Caps are usually not hemispherical in shape, but have a variety of morphologies. SWNTs act as ion channel blockers likely because they are capped.

Carbon nanotubes have tremendous potential applications including transmembrane ion channels, closed reaction chambers, biosensors, materials science and superconductivity, and as slow-release drug delivery vehicles. Attachments of carbon nanotubes to the end of atomic force microscope (AFM) cantilevers would provide crash-proof operation and greater resolution in obtaining images. However, synthesis of open-ended carbon nanotubes (both ends open) with specific diameters and lengths remains a challenge. In addition, functionalized carbon nanotubes containing heteroatoms and/or a non-random arrangement of functional groups are not known.

### BRIEF SUMMARY OF THE INVENTION

This invention provides methods to prepare synthetic carbon nanotubes having controllable properties. The properties which are controllable using the methods provided here include independently and in combination: diameter, length, identity and number of functional groups present and identity and number of heteroatoms present. The synthetic carbon nanotubes prepared using the methods provided herein are open ended (both ends are open). If desired, one or both of the ends can be closed using methods known in the art.

Generally, provided is a method of preparing a synthetic carbon nanotube comprising: providing an aryl ferrocene; forming a cyclopentadienone; reacting the cyclopentadienone with an optionally substituted diphenylacetylene to form a paracyclophane; and cyclodehydrogenating the paracyclophane to form a synthetic carbon nanotube.

More specifically, provided is a method of preparing a synthetic carbon nanotube, comprising: providing an aryl ferrocene; ring-closing and carbonylating the aryl ferrocene to form a ferrocenophane; removing iron and oxidizing the ferrocenophane to form a cyclophane; oxidizing the cyclophane to form a cyclopentadienone; condensing the cyclopentadienone with a benzil to form a cyclopentadienone (in one example, the cyclopentadienone is a tetrakiscyclopentadienone); Diels-Alder cycloadditioning the cyclopentadienone with a diphenylacetylene to obtain a paracyclophane; and cyclodehydrogenating the paracyclophane to obtain a synthetic carbon nanotube.

The cyclopentadienone can be formed using a Grubbs' catalyst, in one embodiment. In one embodiment, the ferrocenophane is formed from reaction of the aryl ferrocene with Fe(CO)₅. In one embodiment, the aryl ferrocene contains from one to three (cyclopentadiene-aryl) groups in a chain. The cyclopentadiene and aryl groups in the (cyclopentadiene-aryl) groups may be attached directly to each other or through the use of a suitable linker or other group. A (cyclopentadiene-aryl) group may be attached to other (cyclopentadiene-aryl) groups directly or through the use of a suitable linker or other group. Linkers are typically an alkylene e.g., —(CH₂)ₙ— or alkenylene (having a C═C double bond in the linker) diradical, where n is an integer indicating the number of repeating units and n is typically small (i.e., 1, 2 or 3) but can be any suitable number. In one embodiment, the aryl ferrocene contains one or more functional groups. In one embodiment, a functional group on the aryl ferrocene is attached to a cyclopentadiene group. In one embodiment, a functional group on the aryl ferrocene is attached to an aryl group. In one example, the one or more functional groups on the aryl ferrocene group are independently selected from the group consisting of: R, halogen, OR, OH, OAc, NR₂, NHAc, SR, O—Si—R₃, and PR₂, wherein the R groups independently may be the same or different and are any desired group including hydrogen; phenyl; substituted phenyl (where the substitutions are independently selected from any suitable group including those listed herein); halogen, including bromine, fluorine or chlorine; C1-C6 alkyl optionally substituted with OR, OH or halogen, including bromine, fluorine or chlorine; diphenyl; and one or more silane-containing protecting groups such as OSi-t-BuMe₂, and any other group which provides the desired functionality as described herein.

In one example, the diphenylacetylene contains one or more functional groups. In one example, the one or more functional groups on the diphenylacetylene are selected from the group consisting of: R, halogen, OR, OH, OAc, NR₂, NHAc, SR, O—Si—R₃, protecting groups such as —OMOM, and PR₂, wherein the R groups independently may be the same or different and are any desired group including hydrogen; phenyl; substituted phenyl where the substitutions are independently selected from any suitable group including those listed herein; halogen, including bromine, fluorine or chlorine; C1-C6 alkyl optionally substituted with OR, OH or halogen, including bromine, fluorine or chlorine; diphenyl; and one or more silane-containing protecting groups such as OSi-t-BuMe₂ and any other group which provides the desired functionality as described herein. In one example, the diphenylacetylene contains one or more heteroatoms independently in the backbone of one or both phenyl rings. In one embodiment, the benzil is optionally substituted using any suitable substituent such as those described herein. In one embodiment, the benzil contains one or more protecting groups such as MOM.

Synthetic carbon nanotubes having controlled properties are also provided. The synthetic carbon nanotubes provided have many uses in a wide variety of fields including medicine, biotechnology, and materials science. The synthetic carbon nanotubes can be used as ion channels for chloride or potassium ions, in the treatment of Cystic fibrosis and other diseases, as semi-conductors, in nanoelectrical devices, for fuel storage systems, and as probe tips in microscopy, for example.

Using the methods provided herein, synthetic carbon nanotubes having specific diameters can be prepared. Some diameters include those between 10 and 25 Å. Some examples of specific diameters include 10 Å, 11 Å, 12 Å, 13 Å, 14 Å, 15 Å, 16 Å, 17 Å, 18 Å, 19 Å, 20 Å, 21 Å, 22 Å, 23 Å, 24 Å and 25 Å. In one particular embodiment, the diameter is 11 Å. In one particular embodiment, the diameter is 22 Å. In one particular embodiment, the diameter is less than 22 Å. In one embodiment, synthetic carbon nanotubes having diameters greater than 22 Å are provided. Larger nanotubes can be prepared by adding additional ferrocenyl moieties in the synthesis, for example, as described herein. In one particular embodiment, the synthetic carbon nanotube has an ion passing diameter (i.e., a diameter that allows a desired ion to pass through). In one particular embodiment, the synthetic carbon nanotube has a calcium passing diameter (i.e., a diameter where calcium ion passes). In one particular embodiment, the synthetic carbon nanotube has a potassium passing diameter (i.e., a diameter where potassium ion passes). In one embodiment, the ion passing diameter is selected to allow the desired ion or ions to pass through the nanotube, but not allow an undesired ion or ions to pass through. When a particular diameter value is given, it is understood that this is an average value. Three specific sizes (diameter×length) of exemplary synthetic carbon nanotubes of the invention are: 10.6 Å×9.7 Å, 21.5 Å×9.7 Å, and 10.6 Å×16.2 Å. When a specific value is given, for example a diameter or length, it is understood that actual measurement is limited by the methods used to determine the value. Presently, computational calculation is used to estimate the diameter and length. The size of the nanotube can be accurately measured using single crystal X-ray analysis. Therefore, it is understood that the specific values listed, for example diameter or length, are ±0.5 Å. All values and ranges within this error are intended to be included in the description to the same extent as if they were specifically listed.

Using the methods provided herein, synthetic carbon nanotubes having specific lengths are prepared. Some lengths include those between 9 and 20 Å. Some examples of specific lengths include 9 Å, 10 Å, 11 Å, 12 Å, 13 Å, 14 Å, 15 Å, 16 Å, 17 Å, 18 Å, 19 Å and 20 Å, or greater, for example. In one embodiment, synthetic carbon nanotubes having lengths greater than 10 Å are provided. In one embodiment, synthetic carbon nanotubes having lengths less than 10 Å are provided. In one embodiment, synthetic carbon nanotubes having lengths greater than 16 Å are provided. When a particular length value is given, it is understood that this is an average value, as described above. The syntheses described herein provide nanotubes with specific lengths and diameters.

All individual combinations of diameter and length are intended to be included in the description to the same extent as if they were specifically listed. All individual values and intermediate ranges of any range given herein are intended to be included herein to the same extent as if the value or range was specifically listed. Specifically, it is intended to be able to add or limit a range or exclude or include an individual value in a claim using the ranges and values provided herein.

Also provided are functionalized synthetic carbon nanotubes. Functionalized synthetic carbon nanotubes contain one or more atoms or bond arrangements which are not present in a non-functionalized synthetic carbon nanotube. One example of functionalized synthetic carbon nanotubes contain one or more non-carbon atoms. These non-carbon atoms may be present in the backbone (tube) structure, such as a heteroatom substitution for carbon, or may be present as a functional group on the structure. Functionalized synthetic carbon nanotubes are useful to tailor the properties of the carbon nanotube to allow the carbon nanotube to have the desired characteristics, such as the ability to interact with biological systems. The carbon nanotube may be functionalized on one or both ends of the carbon nanotube, or may contain functionalizations elsewhere in the structure. If more than one portion of the carbon nanotube is functionalized, any functionalization may be the same or different from other functionalizations on the carbon nanotube. Examples of functional groups include halogens such as F, Cl, and Br; oxygen containing groups such as OR, OAc, OH, CO₂H, CO₂R; metal groups, including Pt and Pd; nitrogen containing groups such as NR₂, NHAc, NH₂, NHCOR, NHSO₂R; sulfur containing groups such as SH, SR and phosphorous containing groups, such as PR₂ and PO(OR)₂, wherein the R groups independently may be the same or different and are any desired group known in the art including hydrogen; phenyl; substituted phenyl where the substitutions are those described herein; halogen, including bromine, fluorine or chlorine; C1-C6 alkyl optionally substituted with halogen, including bromine, fluorine or chlorine; diphenyl; and one or more silane-containing protecting groups such as OSi-t-BuMe₂.

In one embodiment, the functionalized carbon nanotube comprises one or more heteroatoms in the backbone. Any heteroatoms which are present in the carbon nanotube may be the same or different. In one embodiment, the heteroatoms are independently selected from the group consisting of nitrogen, sulfur, phosphorous, and silicon. In one embodiment, the functionalized carbon nanotube comprises one or more nitrogen atoms in the backbone of one end of the carbon nanotube. In one example, the functionalized carbon nanotube has one or more nitrogen atoms in the backbone at one end of the tube, and one or more hydroxyl groups at the other end of the carbon nanotube. In one example, the functionalized carbon nanotube consists of one or more nitrogen atoms in the backbone of one end of the carbon nanotube and one or more carboxylic acid groups at the other end of the carbon nanotube. All combinations of substitutions and functional groups are intended to be included to the extent as if they were specifically listed. Specifically, it is intended to be able to add or exclude a functionalization in a claim using the substitutions and functional groups provided herein.

The synthetic carbon nanotubes of the invention are prepared using the methods described herein, with the appropriate substitutions on the various reactants to produce the synthetic carbon nanotube with the desired properties. As one example, substitution on the diphenylacetylene group is one method to provide functional groups at one or more ends of the synthetic carbon nanotube. As another example, substitution on the aryl ferrocene group provides one method to change the diameter and/or length of the synthetic carbon nanotube. The use of multiple ferrocenyl groups enlarge the diameters of the tubes, for example and use of phenyl rings onto the diphenylacetylene moiety elongate the tubes, for example. The attached functional groups at both ends of the nanotubes can be used to link to various biologically active chemicals for example the anticancer agent, cis-platin.

As used herein, “substituted” or “functionalized” means a group which has one or more atoms which are changed from the unsubstituted or unfunctionalized group. Substitution can mean the replacement of one or more carbon atoms with one or more heteroatoms or the replacement of one or more hydrogen atoms with one or more non-hydrogen atoms. An example of substitution is the replacement of a hydrogen atom with a hydroxyl group. There can be one or more substitutions in a substituted group, and the substitutions can be the same or different. As used herein, an “optionally substituted” group means the group may or may not contain substituted groups. Any group listed may be optionally substituted with any suitable substituent, even if the option of substitution is not specifically mentioned, as long as the substitution does not prevent the group from performing its function, as described herein. Specific examples of groups which may be optionally substituted include independently the aryl ferrocene group, the benzil group and the diphenylacetylene group. As known in the art, any group used may be substituted by a variety of substituents using methods known in the art and performed by one having ordinary skill in the art without undue experimentation. Some substituents are listed herein as examples, although the description is not intended to be limited to those substituents specifically listed.

An “aryl ferrocene” is a compound having at least one ferrocene group

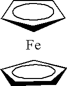

and one or more aryl groups. One example of an aryl ferrocene has the following structure:

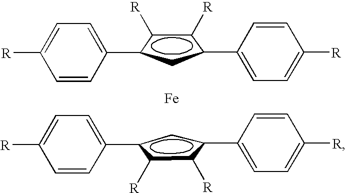

where the R groups independently may be the same or different and are any desired group including: R′, halogen, OR′, OH, OAc, NR′₂, NHAc, SR′, O—Si—R′₃, and PR′₂, wherein the R′ groups independently may be the same or different and are any desired group including hydrogen; phenyl; substituted phenyl where the substitutions are independently selected from any suitable group including those listed herein; halogen, including bromine, fluorine or chlorine; C1-C6 alkyl optionally substituted with OR′, OH or halogen, including bromine, fluorine or chlorine; diphenyl; and one or more silane-containing protecting groups such as OSi-t-BuMe₂R′ groups, and any other group which provides the desired functionality as described herein. It is noted that any available position other than those positions designated as “R” on any part of the group may be substituted. In one embodiment, the aryl ferrocene has the following formula:

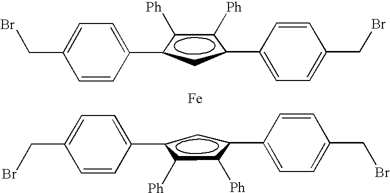

The aryl ferrocene group can have the desired number of cyclopentadienyl-aryl groups in the chain. Adding additional cyclopentadienyl-aryl groups enlarges the diameter of the carbon nanotube formed and lengthens the carbon nanotube formed.

It is noted that every cyclopentadienyl pair does not need to contain an associated iron, as long as the desired ring-closing reaction occurs.

Substitution on the aryl ferrocene provides one way to obtain functional groups on one or more ends of the carbon nanotube. One example of this is shown below, where the substitution of the —OMOM (O-methoxymethyl) group on the aryl ferrocene provides one method to obtain hydroxyl functional groups on one end of the carbon nanotube.

A cyclophane is a compound having an aromatic unit and an aliphatic chain that forms a bridge between two non-adjacent positions of the aromatic ring. A paracyclophane is a cyclophane with at least two groups in the “para” position.

A ferrocenophane group is a cyclized ferrocene-containing group.

As used herein, a diphenylacetylene group has the following formula:

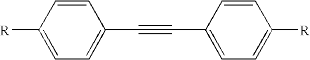

where the R groups independently may be the same or different and are selected from the group consisting of suitable substituents, including R′; halogen; NR′₂; NHAc; O—Si—R′₃; protecting group such as —OMOM; OAc; OH; OR′; SR′; PR′₂; wherein the R′ groups independently may be the same or different and are any desired group including hydrogen; phenyl; substituted phenyl where the substitutions are independently selected from any suitable group including those listed herein; halogen, including bromine, fluorine or chlorine; C1-C6 alkyl optionally substituted with OR′, OH or halogen, including bromine, fluorine or chlorine; diphenyl; and one or more silane-containing protecting groups such as OSi-t-BuMe₂R′ groups, and any other group which provides the desired functionality as described herein. Although the “R” substituents are shown in the para position in the structure, this is not the only useful or possible configuration. For example, one “R” may be in the para position and the other may be in the meta position. In addition, there may be one or more substitutions on one or more rings of the diphenylacetylene group. The substitutions are independently selected from any suitable substituent, including those described above. The ring structures of diphenylacetylene may be optionally substituted with one or more heteroatoms, such as nitrogen atoms in the ring.

Benzil is the following compound:

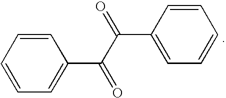

In the methods described herein, benzil may be optionally substituted. Some examples of optional substitution on the benzil group include protecting groups attached to the phenyl ring. In one example, the benzil contains one or more protecting groups, which in one example is MOM (methoxymethyl). A protecting group may be optionally substituted, such as with a halogen (for example, MOM-Cl). The protecting group or other substituent may be attached to the benzil group using any suitable linker, such as —O— or —CH₂—O—, or other linkers, as known in the art. Other protecting groups and linkers may be used, as known in the art. Other examples of substitutions on the benzil group are one phenyl group attached in the para position on one ring, and another phenyl group attached in the meta position on the other ring.

### BRIEF DESCRIPTION OF THE DRAWING

FIG. 1 shows AFM images of protofibrils (upper panel), small oligomers (middle panel), and an expansion of a small oligomer (lower panel) of Aβ42 obtained from a Nanoscope IIIa SPM atomic force microscope (Digital Instruments, Inc. Santa Barbara, Calif.) with tapping mode using a high aspect ration tip (Veeco Nanoprobe™ tips, Model TESP-HAR).

### DETAILED DESCRIPTION OF THE INVENTION

The following description is intended to be exemplary and provide non-limiting examples of some embodiments of the invention. As is known in the art, it is understood that the same compounds and compositions can be named differently and can be represented differently in a formula by those of ordinary skill in the art. Therefore, when a compound is named or a formula shown in the disclosure herein, all equivalent names or formulas are intended to be included. As known in the art, different compounds that have the equivalent function to another compound can be used in organic synthesis. These equivalents are intended to be included in this description.

Chemical synthesis methods to synthesize carbon nanotubes having desired diameters, lengths and compositions, including the presence of heteroatoms and functional groups have been developed. Scheme 1 illustrates three examples of synthetic carbon nanotubes of the invention:

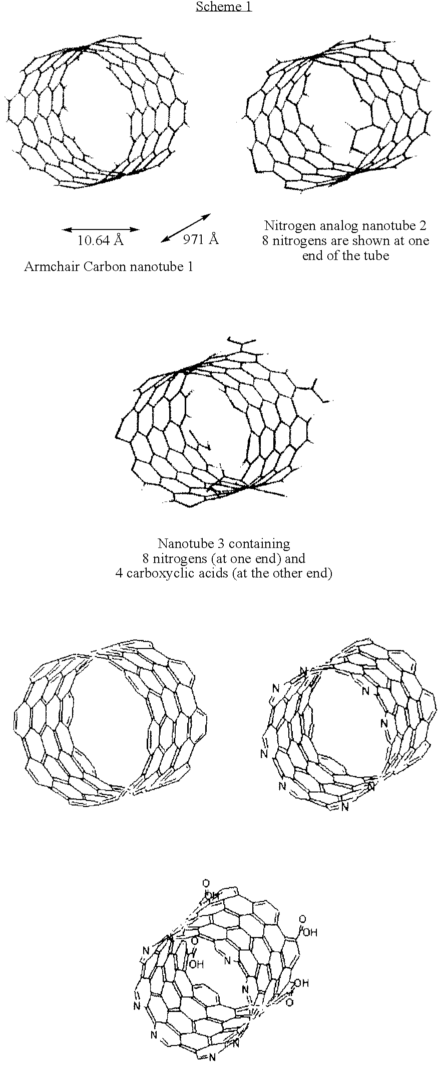

The diameter and length of carbon nanotube 1 are 10.64 and 9.71 Å, respectively (from Chem3D, molecular mechanics computation). Nanotube 2 contains eight nitrogens at one open end of the tube in an alternate fashion (equivalents to four bipyridyl moieties). Nanotube 3 consists of eight nitrogens on one side of the tube and 4 carboxylic acid groups on the opposite side of the tube. Each of the four carboxylic acids and each of the bipyridyl moieties are tightly bonded through hydrogen bond donor and acceptor combinations.

Synthesis of Functionalized Single-Walled Carbon Nanotubes with Specific Diameters and Lengths

A retrosynthesis of nanotube 1 is shown in Scheme 2, in which a synthetic intermediate of 1, substituted all-Z-[0₈]paracyclophane (23; vide infra, Scheme 5), is produced from belt-like compound 8. This cyclic compound 8 is synthesized from a condensation of benzil and diketone 9. The formation of macrocycles from acyclic precursors produces large amounts of oligomers. This problem is avoided in these methods by using a ferrocene moiety as the anchor for the ring closing reaction. Hence, compound 10 and analogous compounds are the synthetic targets. These targets are prepared from tetrabromide 12. Compound 12 can be derived from cyclopentadienone 13, which in turn are produced from bromide 14 from a bis-coupling reaction with Fe(CO)₅ followed by condensation with benzil. Overall, a repetitive carbonylation with Fe(CO)₅ and condensation with benzil are used to construct the cyclopentadienone moieties.

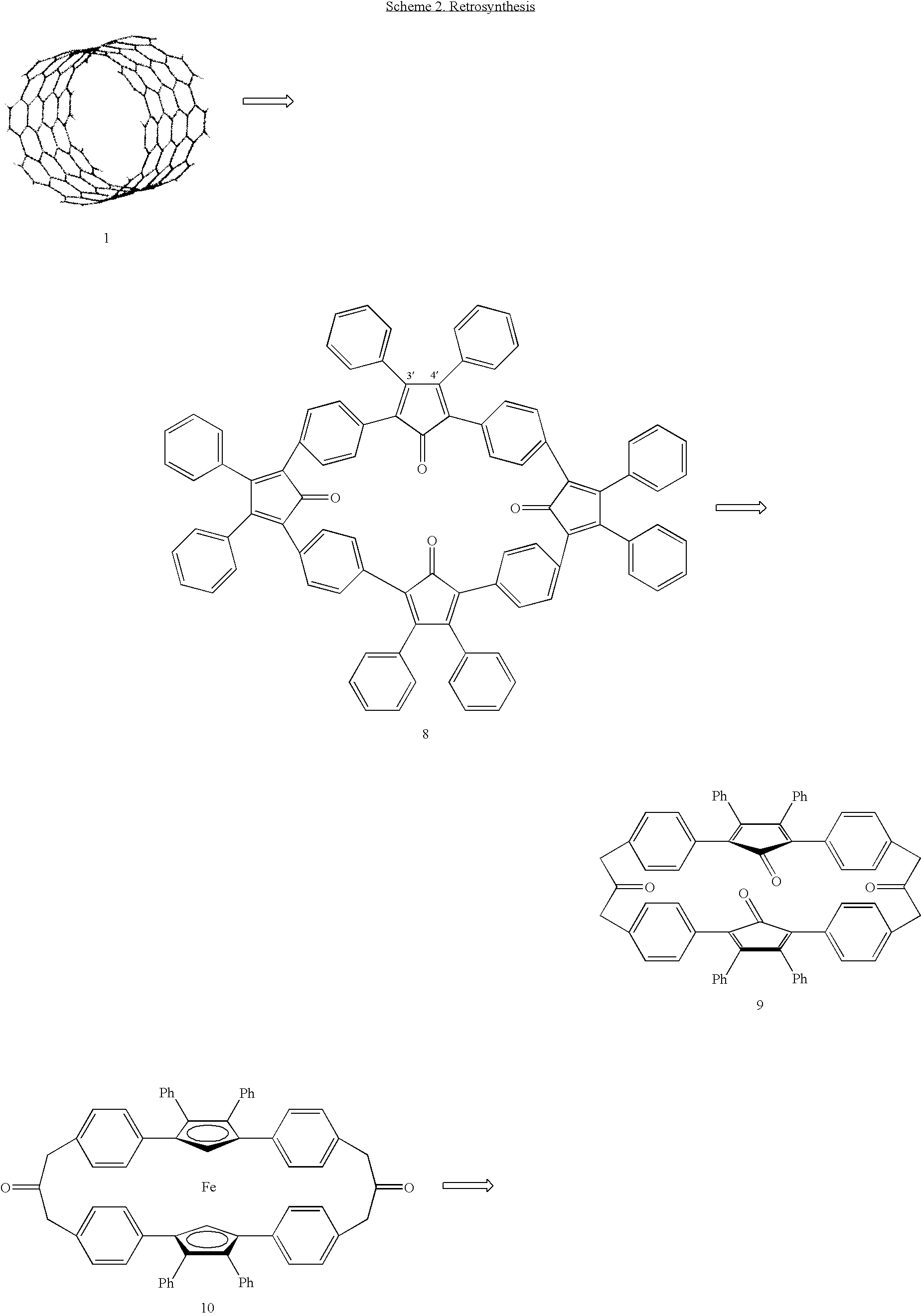

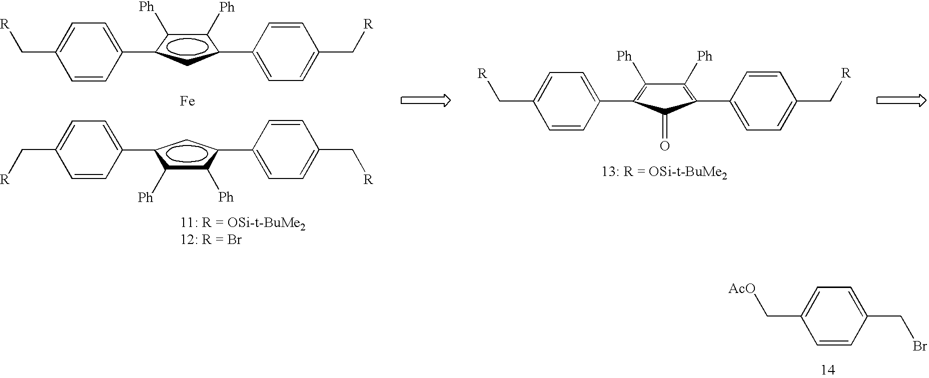

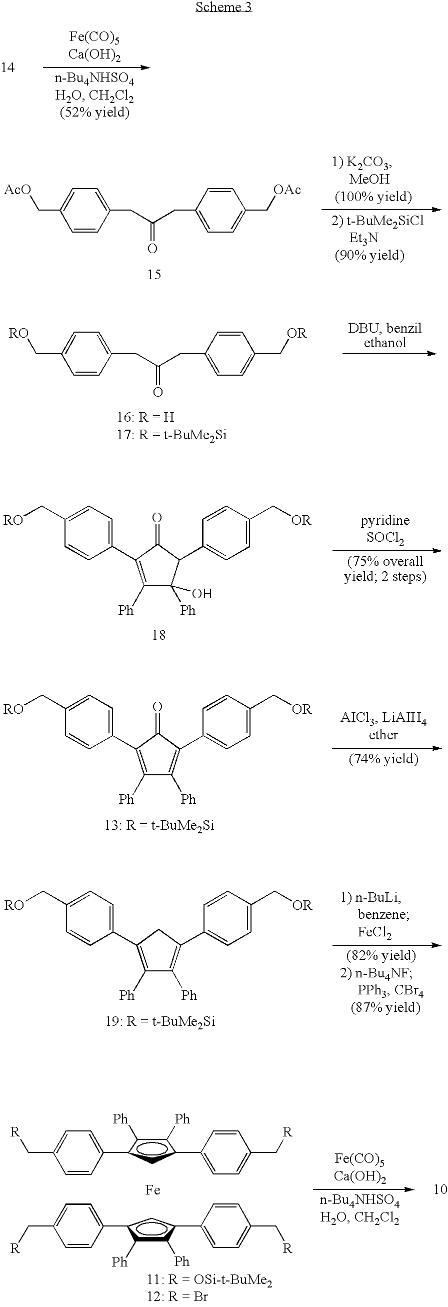

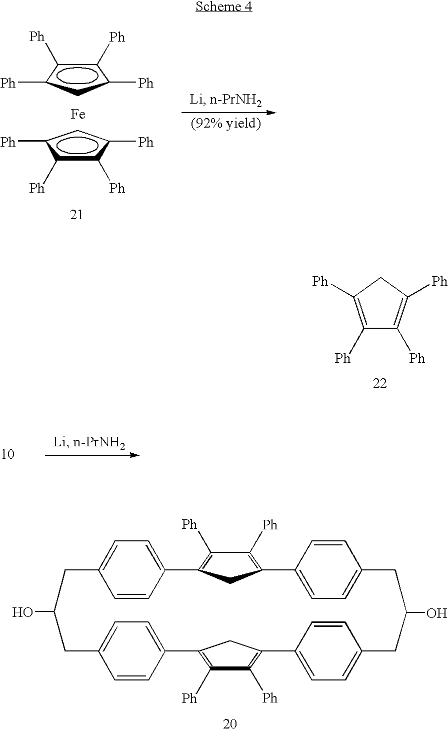

Tetrabromide 12 has been synthesized by a simple route outlined in Scheme 3. 4-Bromomethylbenzyl acetate (14) was obtained from a modified procedure of the reported method²⁸ in 58% yield using 1,4-(bisbromomethyl)benzene and KOAc in CH₃CN. Iron pentacarbonyl mediated carbonylation²⁹ of bromide 14 with Fe(CO)₅—Ca(OH)₂-n-Bu₄N⁺HSO₄⁻ in dichloromethane and water at 25° C. gave ketone 15 (52% yield). Basic hydrolysis of 15 with potassium carbonate in methanol followed by silylation with t-butyldimethylsilyl chloride, triethylamine, and 4-(dimethylamino)pyridine (DMAP) in dichloromethane furnished a 90% yield of bis-silylether 17. Condensation of 17 with benzil and 1,8-diazabicyclo[5.4.0]undec-7-ene (DBU)³⁰ in ethanol followed by the treatment of the resulting alcohol 18 with thionyl chloride in pyridine afforded cyclopentadienone 13 (75% yield in two steps).

Compound 18 was carried out in the next step without purification. Reduction of 13 with aluminum hydride, derived from AlCl₃ and lithium aluminum hydride,³¹ in ether gave cyclopentadiene 19 (74% yield), which was converted to ferrocene 12 by the treatment of n-BuLi in benzene-hexane and FeCl₂ in THF³¹ followed by desilylation with n-Bu₄NF in THF and bromination with PPh₃—CBr₄. Tetrabromide 12 is converted into ferrocenophane 10 with Fe(CO)₅, Ca(OH)₂, and n-Bu₄N⁺HSO₄⁻ in water and dichloromethane. Spectral data, NMR and mass spectra, of the newly synthesized materials agreed with the assigned structures. For example, ¹H NMR spectrum of tetrasilyl ether 11 (a red crystalline material) shows signals at δ7.1−6.8 (m, 36H, Ar), 5.47 (s, 2H, Cp—H), 4.62 (s, 8H, CH₂), 0.95 (s, 36H, t-Bu), 0.09 (s, 24H, MeSi) ppm, and the mass spectrum exhibited the M⁺peaks at 1370.60 (M⁺, 100%), 1371.40 (M+1⁺, 100%), and 1372.20 (M+2⁺, 50%), which confirm the structural assignment.

After the formation of the ferrocenophane 10, the iron is removed from the ferrocene moiety. A model compound, octaphenylferrocene (21), was used for the studies, and reduction of ferrocene 21 with lithium in n-propylamine^{32a} at 25° C. gave an excellent yield (91%) of 1,2,3,4-tetraphenylcyclopentadiene (22) (Scheme 4). Syntheses of compounds 10 and 20 are described below. It is believed that except for compound 14, compounds 11-19 have not been described previously.

The silyl ether protecting groups of cyclopentadienone 13 have been removed, and the resulting diol has been brominated with 2 equivalents each of Ph₃P and carbon tetrabromide to produce the corresponding dibromide. Cyclization of this dibromide is carried out to compare the yields of this reaction and that of the above conversion of 12 to 10.

This eight-step synthesis of ferrocene 12 and the conversion to compound 20 constitute a versatile method for the construction of various sizes and functionalized nanotubes and heteroatom-containing nanotubes. The diameter and the length of the nanotubes are expanded by simple modifications of the carbonylation protocol [Fe(CO)₅] and substituted benzils and diarylacetylenes. These modifications and the conversion of 12 to nanotube 1 are described below.

Compound 12 serves as a key intermediate in the synthesis of armchair carbon nanotube 1 (Scheme 5). First, tetrabromide 12 is converted into cyclophane 20 by the treatment with Fe(CO)₅, Ca(OH)₂, and n-Bu₄NHSO₄ in a diluted solution of dichloromethane and water (Scheme 3) followed by reduction with lithium in n-propylamine (Scheme 4). A similar carbonylation reaction appears in the conversion of 14 to 15, and the ferrocene moiety serves as an anchor to facilitate the ring forming reaction. The ease of forming 1,1′,3,3′-bis(trimethylene)ferrocene supports the anchor effect of ferrocene.^{32b} The diluted solution prevents the formation of dimers or oligomers. The reductive removal of iron from ferrocene 21 to cyclopentadiene 22 supports the deiron reaction of 10 to 20. The reported oxidation of tetraarylcyclopentadienes to tetraarylcyclopentadienones³³ with a sequence of reactions of p-nitroso-dimethylaniline in methanol-toluene and HCl (to remove the resulting hydrazone) is used to study the oxidation of 20 to cyclopentadienone 9. Treatment of 20 with p-nitroso-dimethylaniline followed by HCl and oxidation of the dihydroxyl functions with iodoxybenzoic acid (IBX) in DMSO³⁴ produces compound 9. Although the hydroxyl function does not react with p-nitroso-dimethylaniline, if the hydroxyl groups of 20 react with the reagents used, they are protected with acetic anhydride and pyridine as acetoxy, and are removed after oxidation of the cyclopentadienyl ring with K₂CO₃ in MeOH. Alternatively, the cyclopentadienyl moieties of 20 can be hydroxylated with 4 equivalents of n-BuLi (2 eq. are used to deprotonate the dihydroxyl functions) followed by bis(trimethylsilyl)peroxide,³⁵ and the resulting tetrahydroxyl intermediate is oxidized with IBX-DMSO. Aldose condensation of diketone 9 with benzil and DBU followed by thionyl chloride in pyridine³⁰ (see below) provides tetrakiscyclopentadienone 8. Diels-Alder cycloaddition of cyclopentadienone 8 with 4 equivalents of diphenylacetylene under refluxing diphenyl ether affords all-Z-[0₈]paracyclophane 23. Cyclodehydrogenation of 23 with ferric chloride in nitromethane and dichloromethane²⁷ furnishes armchair carbon nanotube 1.

It should be noted that compound 8 may have two major conformers (from the restricted rotation of C—C sigma bonds of the cyclophane ring system); one with all four carbonyl groups pointing inside the macrocycle and the one with four carbonyls pointing outside of the macrocycle. From molecular modeling and computational calculations, the conformer with carbonyl groups pointing inside the macrocycle is the most stable conformer; while the other would have a large repulsion from C3′- and C4′-phenyl rings of the cyclopentadienone moieties with the remaining inert moieties of the macrocycle. The rotational barriers of sigma bonds between the macrocycle phenyl rings and cyclopentadienone rings should be large, because in order to rotate (360°), the two C3′ and C4′-phenyl rings of the cyclopentadienone would have to pass through the inert part of the cyclophane ring system. Hence, it is expected that only one conformer of compound 8 is formed and is depicted in Scheme 6. The structure is obtained from Chem3D (molecular mechanics computation), and a bowled belt-like structure is found. Although fifteen steps are needed for the synthesis of nanotube 1 starting from 4-bromomethylbenzyl acetate (14), the sequence of reactions is relatively straightforward, and yields of most steps are high. The final seven steps (from compound 12 to 1) have literature precedents.

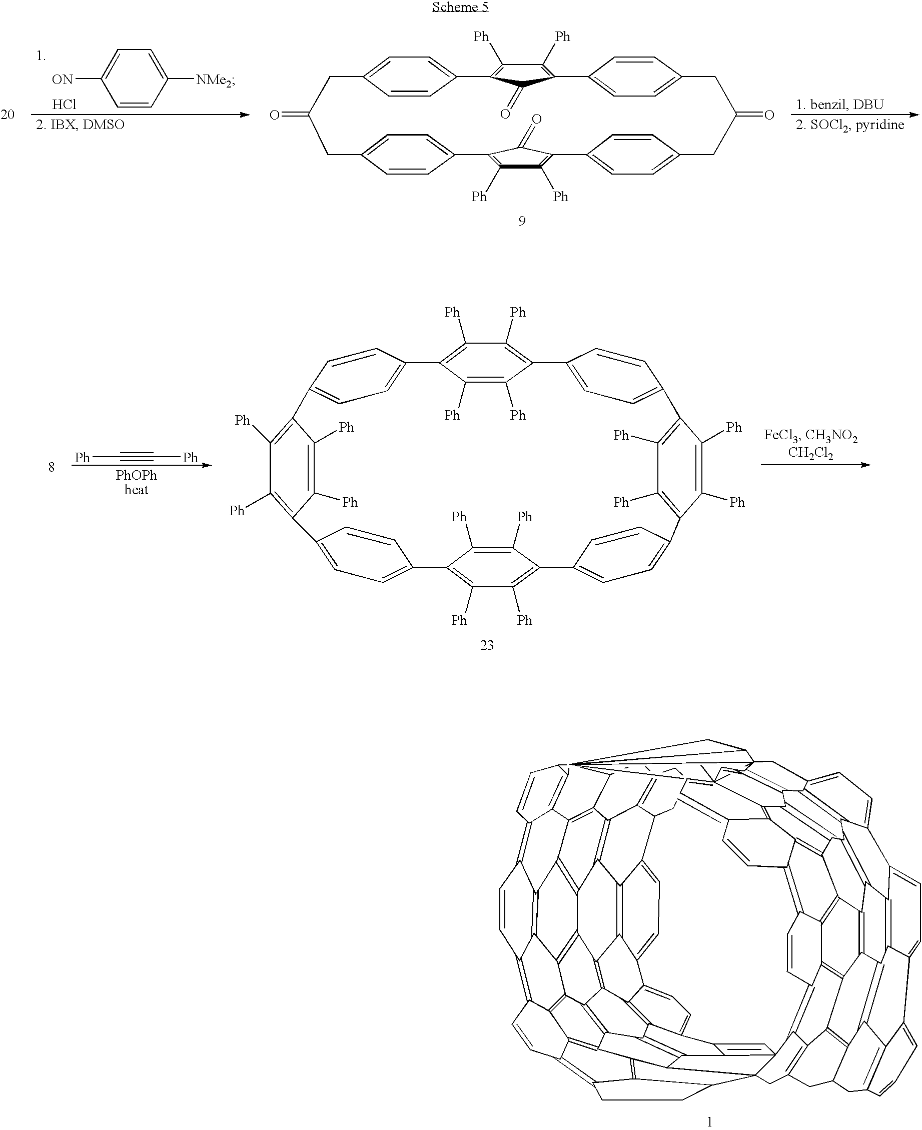

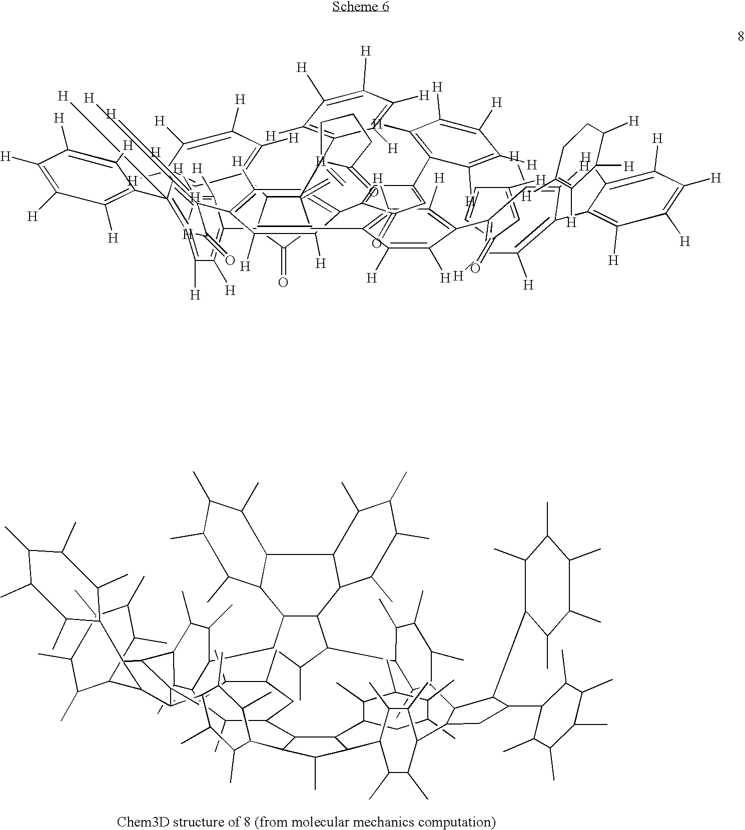

### Alternative Synthesis Method

The reaction of compound 12 (see Scheme 3) with Fe(CO)₅, Ca(OH)₂, and n-Bu₄NHSO₄ in H₂O and dichloromethane produce a mixture of unidentifiable materials along with the desired product 10 (identified through mass spectrometry). An alternative pathway leading to compound 23 was developed using Grubbs' catalyst (a commercially available reagent) to cyclize the ferrocene. Schemes 7 and 8 show formations of diferrocenocyclophane 77 and monoferrocenocyclophane 78 as key intermediates for constructions of nanotube 1 and its larger diameter carbon nanotube.

Desilylation of compound 11 with tetra-n-butylammonium fluoride in THF gave tetraol 74, which was not purified and treated with IBX in DMSO to give tetraaldehyde 75 in 99% yield (2 steps). Treatment of 75 with 6 equivalents of vinylmagnesium bromide in THF afforded a 60% yield of tetraene 76, which was subjected to Grubbs' second generation catalyst in 10⁻³ M of benzene at 50° C. gave diferrocenocyclophane 77. Compound 77 was identified by mass spectrometry, which spectrum showed a mass of 1925.74 (M⁺). The relatively concentrated solution led to the formation dimer 77. It should be noted that dimer 77 is used to synthesize larger diameter carbon nanotubes (see similar procedures described in Scheme 8 leading to carbon nanotube 1 from monoferrocenocyclophane 78).

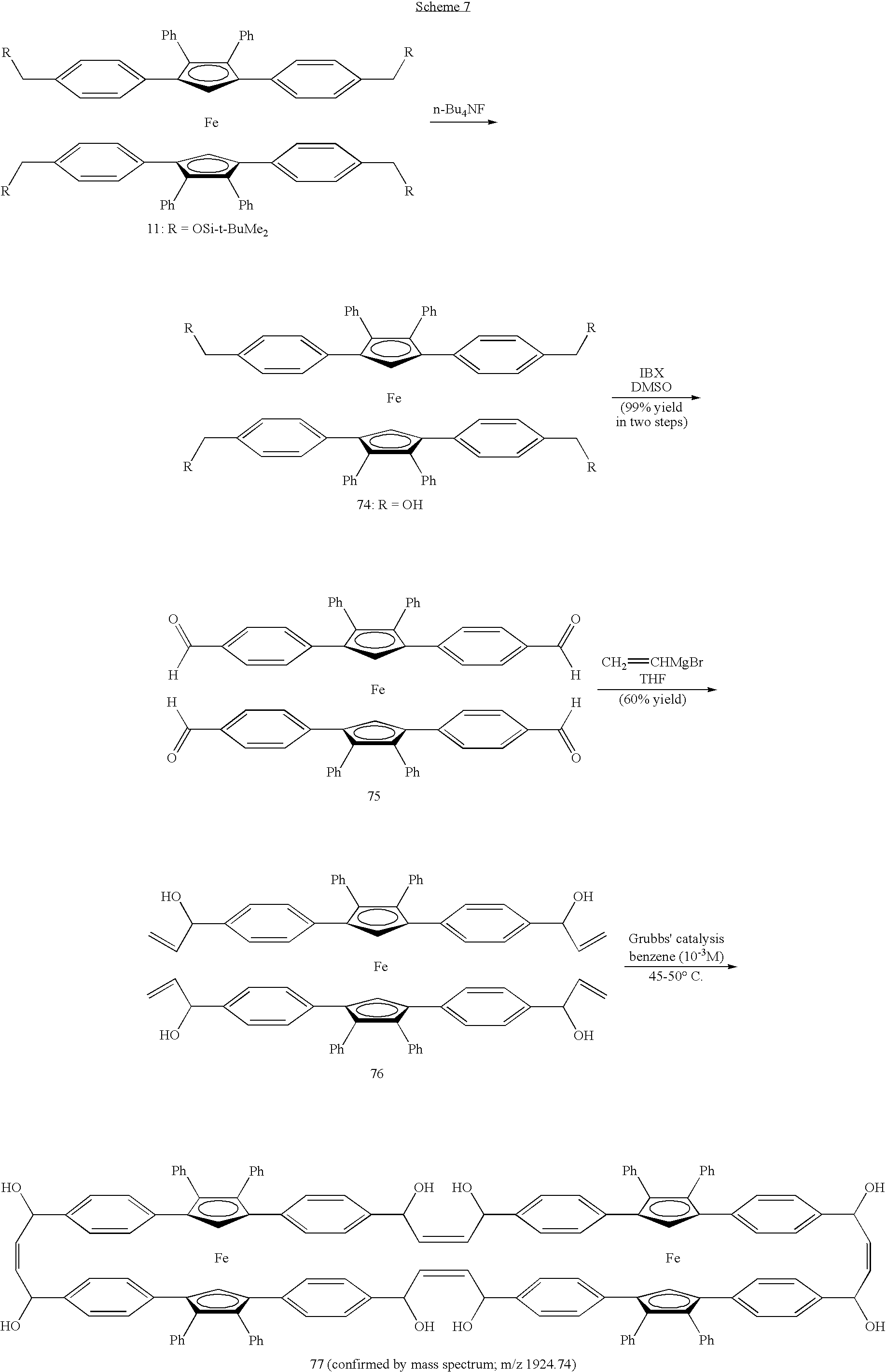

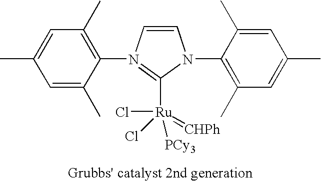

Under less concentrated benzene solution, 10⁻⁴M, olefin metathesis of compound 76 provided ferrocenocyclophane 78 as the major product [MS m/z 962.219 (M⁺), 963.224 (M+1), 964.222 (M+2)], which is converted to carbon nanotube 1 (Scheme

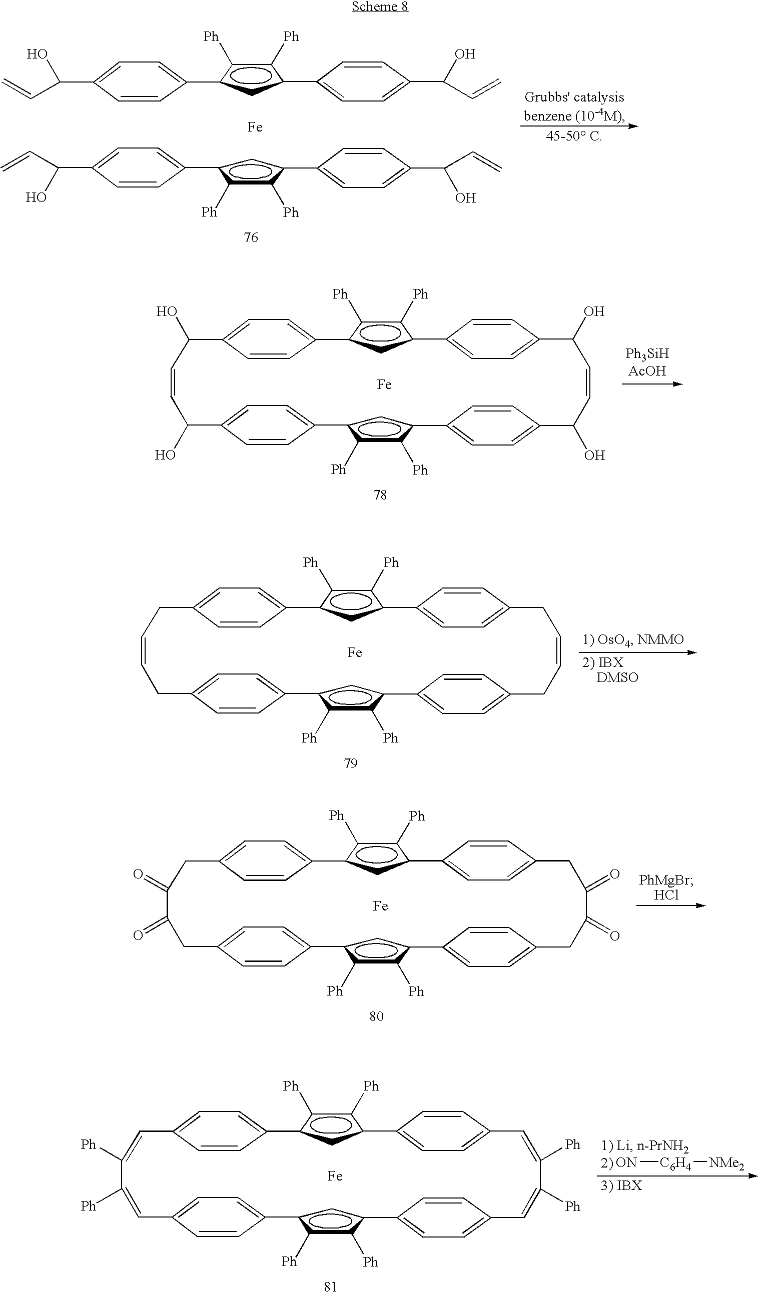

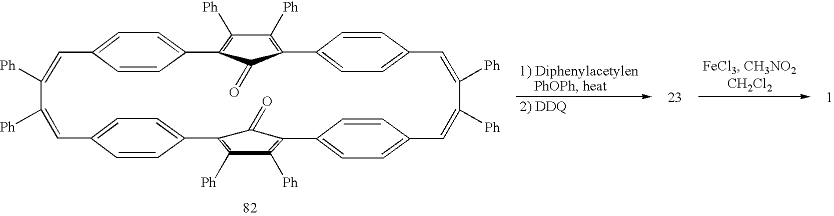

8). Dehydroxylation of 78 with triphenylsilane and acetic acid⁷⁵ provides diene 79, which is dihydroxylated with osmium tetroxide and N-methylmorpholine-N-oxide in t-butanol, acetone and water followed by oxidation with IBX in DMSO to furnish tetraone 80. Addition of phenylmagnesium bromide in THF followed by dehydration with HCl gives tetraene 81, which deironized and oxidized (see Scheme 5) to afford cyclopentadienone 82.

Quadruple Diels-Alder reactions of 82 with diphenylacetylene in diphenyl ether under reflux followed by dehydrogenation with 2,3-dichloro-5,6-dicyano-1,4-quinone (DDQ) furnishes beltene 23. Cyclodehydrogenation of 23 with ferric chloride in nitromethane and dichloromethane provides carbon nanotube 1.

### Synthesis of Functionalized Carbon Nanotubes:

A major deficiency in the carbon nanotube preparation methods currently used is that attempts to functionalize carbon nanotubes result in randomized reactions and possible functionalization reactions are limited to a few oxidation reactions. Carboxylic acids can be introduced into the nanotube, but the exact locations and amounts are undetermined and unpredictable. A sidewall protection of MWNTs using a polystyrene matrix followed by a plasma oxidation of the carbon nanotube tips to carboxylic acid functionalities has recently been reported. However, the number of carboxylic acid groups and locations on the MWNTs remained unidentifiable. Hence, selective introduction of various functional groups, such as Br, OH, SH, NH₂, and CO₂H groups, will enhance the ability of connections with other materials and solubilizing groups. The syntheses of these analogs are readily be carried out using the methods described here, using different diarylacetylenes, compounds 24-26, in the Diels-Alder reactions with compound 8.

Bis(4-bromophenyl)acetylene (24) is prepared from 4-iodobromobenzene, acetylene, bis(triphenylphosphino)palladium dichloride, CuI, and piperidine,³⁸ acetylene 25 from 4-acetoxy-iodobenzene, bis(tributylstannyl)acetylene, tetrakis(triphenylphosphino)palladium, LiCl, and a catalytic amount of 2,6-di-t-butyl-4-methylphenol in dioxane,³⁹ and compound 26 from bromination of p,p′-dinitrostilbene with Br₂, dehydrobromination with KOH, reduction of the nitro functions with Rupe's N₁ and H₂, and acetylation with acetic anhydride.⁴⁰ Diels-Alder reactions of 8 with 4 equivalents of acetylene 24, 25, and 26, separately in refluxing diphenyl ether produce cyclophanes 27, 28, and 29, respectively (Scheme 9). As mentioned above, the bowled belt-like structure of 8 (Scheme 6) would prevent Diels-Alder reactions taking place in the inert part of the macrocycle. Consequently, diarylacetylenes approach the cyclopentadienone moieties of 8 from the outside face of the macrocycle. Hence, only one regioisomer is expected from the Diels-Alder reaction. The decarbonylation at elevated temperature generates paracyclophanes 27-29. Although unlikely, it is possible that regioisomers can be formed from the Diels-Alder reactions. The regioisomers, if formed, are separated by either silica gel column chromatography or HPLC, and their structures are identified by single-crystal X-ray analysis. The paracyclophanes are expected to be crystalline materials. Cyclodehydrogenation of 27-29 separately with FeCl₃ in nitromethane and dichloromethane at 25° C. affords functionalized nanotubes 30, 31, and 32, respectively. It should be noted that both p,p′-disubstituted diarylacetylenes and m,m′-disubstituted diarylacetylenes provide the same octasubstituted carbon nanotubes and either can be used. The m,m′-substituted diarylacetylenes have also been reported.³⁸⁻⁴⁰ Basic hydrolysis of octaacetate 31 with K₂CO₃ in methanol gives octahydroxy derivative 33, and hydrolysis of octaamide 32 with KOH in H₂O and diglyme provides octamino nanotube 34. Finally, displacement of octabromo analog 30 with α-(trimethylsilyloxy)propylthiol and n-BuLi⁴¹ followed by desilylation with KF gives thiol 35. These compounds are used in the biological application of nanotubes (see below).

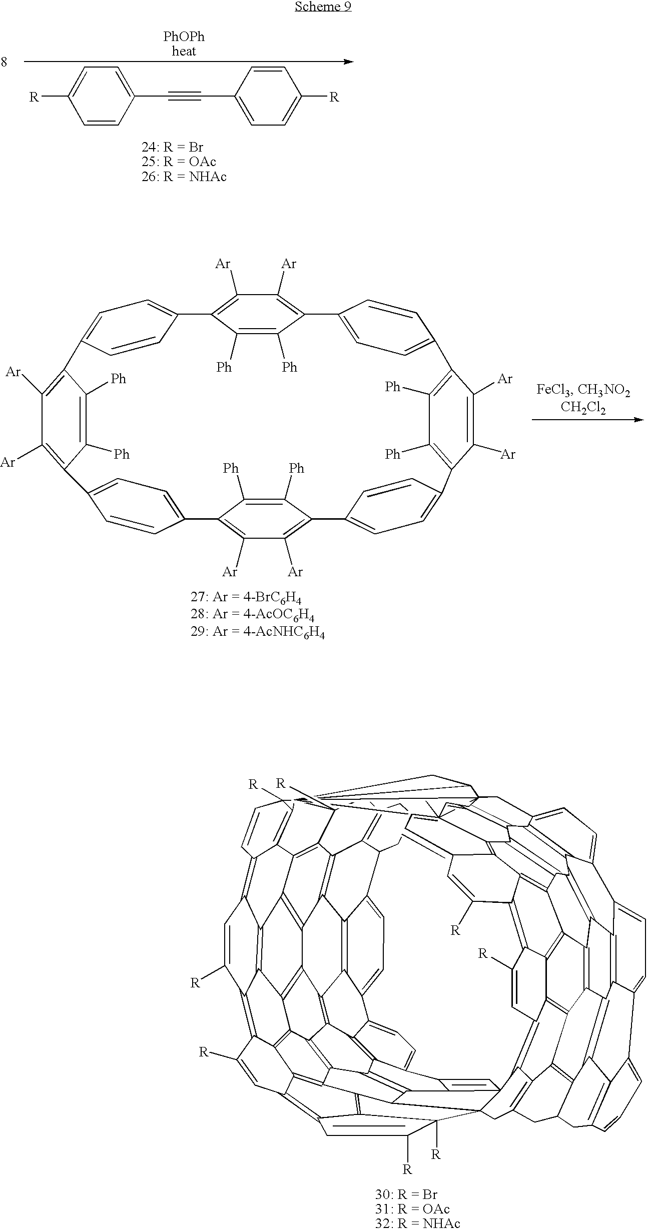

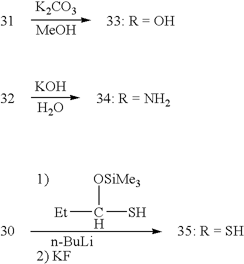

It is also possible to synthesize two different kinds of functional groups on two open ends of the nanotube. As one example, Scheme 10 illustrates the synthesis of a carbon nanotube containing eight hydroxyl groups at one end and eight bromines at the other end of the tube (ie., compound 40). Condensation of ketone 17 (see Scheme 3) with 4,4′-di(methoxymethyloxy)benzil (36), derived from alkylation of 4,4′-dihydroxybenzil⁴² with NaH and methoxymethyl chloride (MOM—Cl; MOM═MeOCH₂—), and DBU followed by thionyl chloride in pyridine provide cyclopentadienone 37.

The conversion of compound 37 to compound 38 is similar to that from 13 to compound 8 (see Schemes 3-5) by the sequence: (i) reduction of the carbonyl function with aluminum hydride; (ii) formation of ferrocene moiety with n-BuLi followed by FeCl₂; (iii) desilylation accompanied by bromination; (iv) ring closure with Fe(CO)₅, Ca(OH)₂, and n-Bu₄NHSO₄ in water and dichloromethane; and (v) condensation with benzil 36 followed by dehydration with thionyl chloride in pyridine. Compound 38 has a similar bowled belt-like structure as that of compound 8, and Diels-Alder reaction with 4 equivalents of acetylene 24 in refluxing diphenyl ether provides paracyclophane 39, which upon oxidation with FeCl₃ furnishes nanotube 40. Removal of the MOM protecting group of compound 40 with BF₃.ether and ethanethiol⁴³ in dichloromethane gives octabromo-octahydroxylnanotube 41.

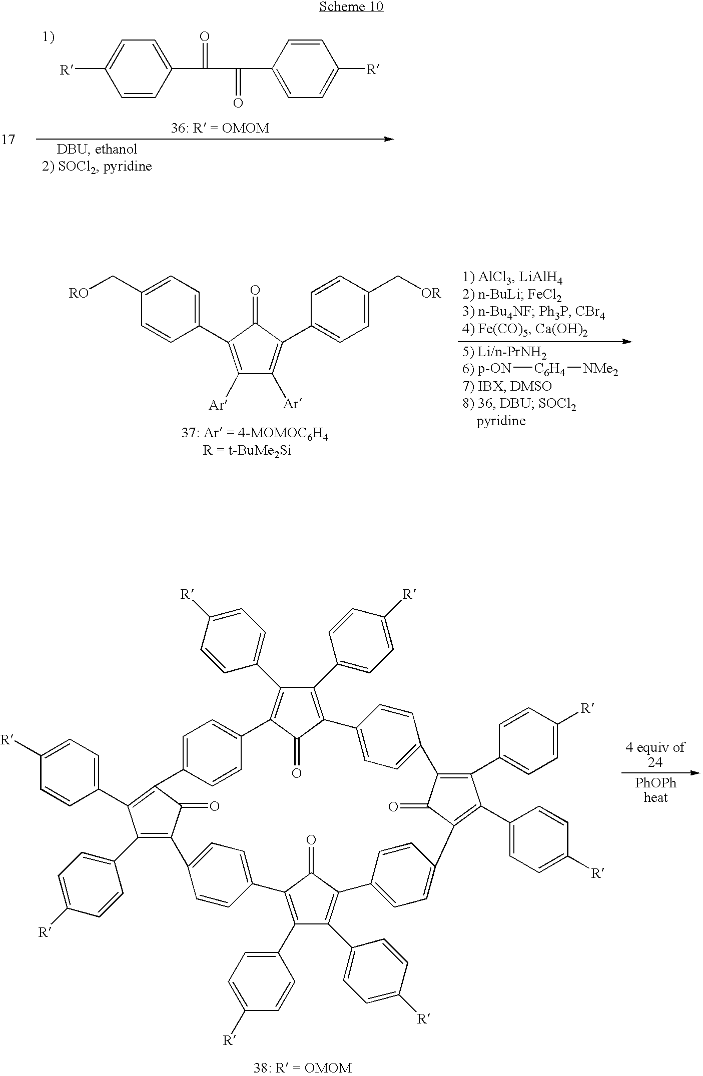

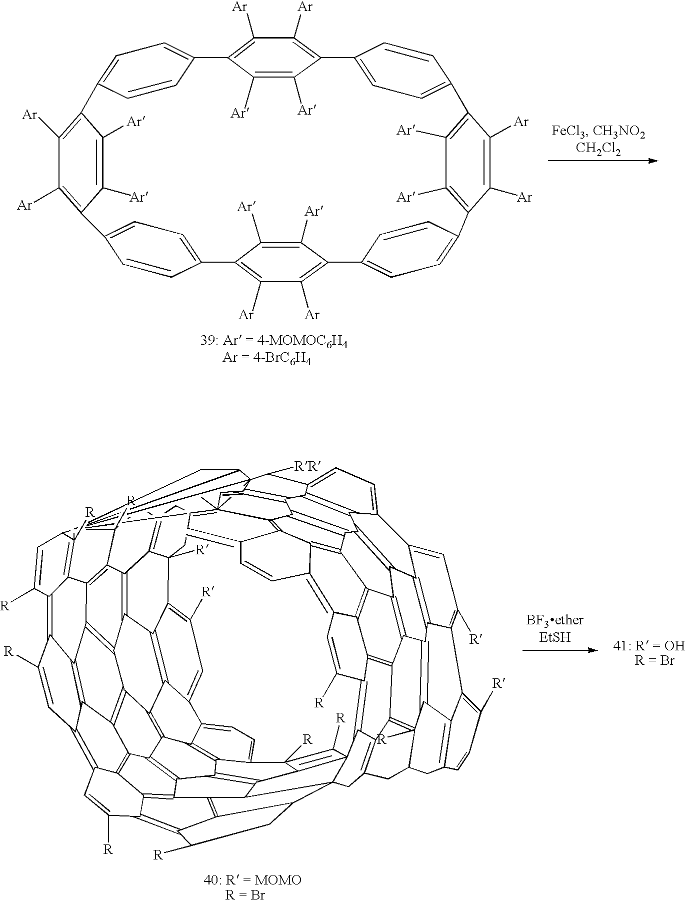

Carbon nanotubes and functionalized derivatives are thus synthesized through a straightforward sequence of reactions. The macrocyclic ring closing reactions utilizing ferrocenyl moieties as an anchor to facilitate the annulation serves as a key step in the construction of nanotubes and is a general method for the construction of macrocycles. Moreover, the octabromo nanotube 30 can be used to introduce various functional groups via Suzuki coupling reaction, displacement reaction, or formation of the Grignard reagent followed by the reaction with electrophiles, to name a few.

The synthesis of nanotube 3 containing ammonium ions on one end and carboxylate ions on the other end, is described in Scheme 12. This nanotube can be used as an ion channel or drug delivery molecule, for example.

### Synthesis of Heteroatom-Containing Nanotubes:

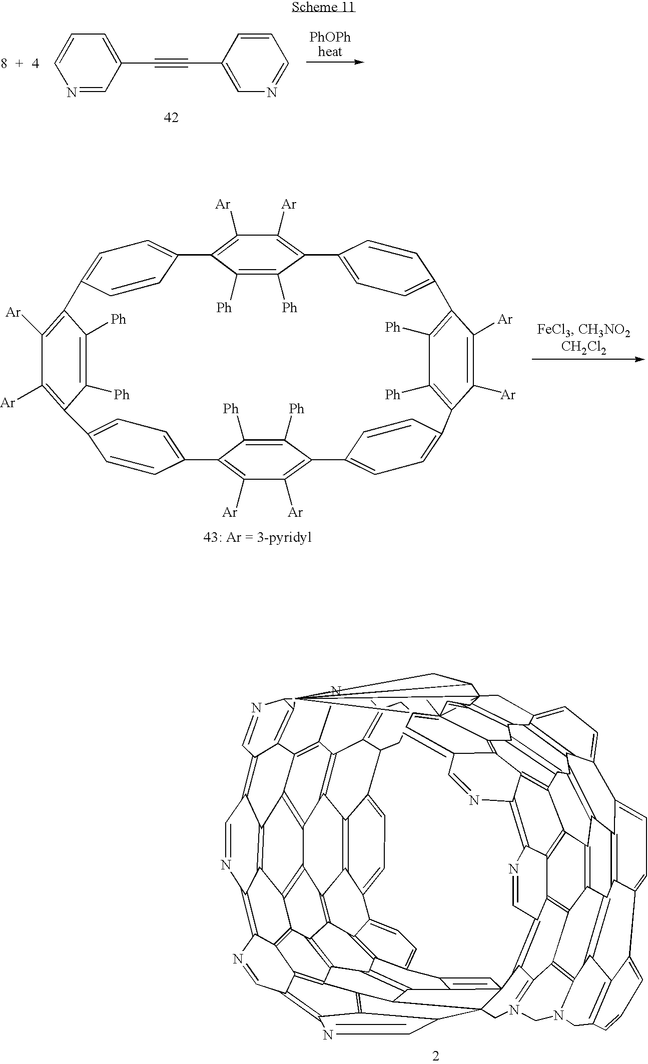

It is unlikely that current reported methods¹⁷⁻²² of the preparations of MWNTs and SWNTs could provide heteroatom-containing carbon nanotubes such as compounds 2 and 3 (see Scheme 1). Heteroatom containing nanotubes will provide new materials not only for biological and material applications (vide infra), but also their physical properties and spectroscopy. The synthesis of nanotube 2 is readily carried out from the Diels-Alder reaction of macrocycle 8 and 4 equivalents of bis(3-pyridyl)acetylene (42)⁴⁴ in refluxing diphenyl ether to give paracyclophane 43 (Scheme 11). The preparation of acetylene 42 from 3-bromopyridine, Pd(OAc)₂, 1,4-bis(diphenylphosphino)butane, KOH, 18-crown-6, and 1-bromo-2-chloroethane in 61% yield has been reported.” Cyclodehydrogenation of paracyclophane 43 with ferric chloride in nitromethane and dichloromethane provides nitrogen-nanotube 2.

The synthesis of bifunctional nanotube 3 is similarly carried out from macrocycle 9 and benzil 44 (Scheme 12). Benzil 44 is prepared from methyl 4-formylbenzoate by the sequence of reactions: (i) protection of the aldehyde function with N-lithiomorpholine followed by trimethylsilyl chloride and then reduction of the ester function with lithium aluminum hydride;⁴⁵ (ii) alkylation with 2 equivalents of NaH and 2 equivalents of methoxymethyl chloride; (iii) benzoin condensation with sodium cyanide in aqueous ethanol; and (iii) oxidation of the resulting benzoin with IBX. Condensation of macrocycle 9 with benzil 44 and DBU followed by thionyl chloride and pyridine give macrocycle 45. Diels-Alder cycloaddition of 45 with 4 equivalents of acetylene 42 in refluxing diphenyl ether affords paracyclophane 43, which upon treatment with FeCl₃ in nitromethane and dichloromethane, removal of the MOM protecting group with BF₃.ethereal-ethanethiol, and oxidation of the arylmethanol with pyridinium dichromate (PDC) in DMF at 25° C.⁴⁶ furnish bifunctional nanotube 3. Since nanotube 3 contains both basic pyridine and acidic carboxyl moieties, it is stored as hydrochloric acid salt.

Nitrogen-containing carbon nanotubes including carboxylic acid functions, such as compounds 2 and 3, are synthesized similarly from the methods described herein. These compounds can be used as bases and for self-assembling and inclusion materials. The application is described below.

### Synthesis of Larger Size Nanotubes and Self-Assembly of Nanotubes.

It is apparent that a number of modifications can be applied in the nanotube synthesis described herein to generate larger diameter and greater length of nanotubes. The enlargement of the diameter and lengthening of the tube is carried out by extending a greater number of ferrocenyl-phenyl moieties and the uses of substituted benzils and diarylacetylenes. As examples of this concept, the following schemes illustrate several modifications leading to 0₁₆; (4,3)-armchair nanotube 47 and 0₈; (7,6)-armchair nanotube 48 (The 4,3 and 7,6 numberings indicate the numbers of stacked aromatic rings along the tube, and do not follow the m,n designations in carbon nanotubes).

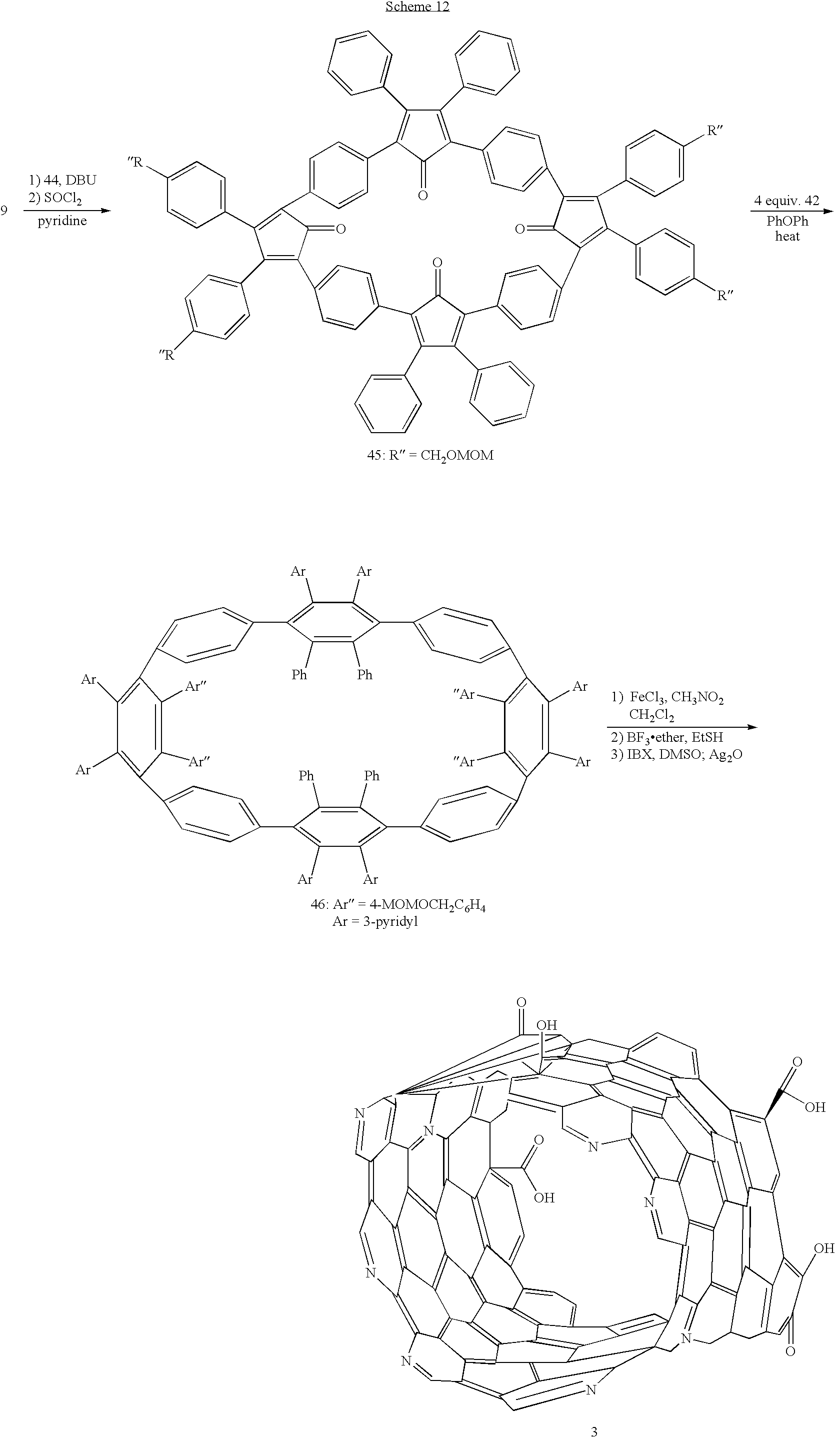

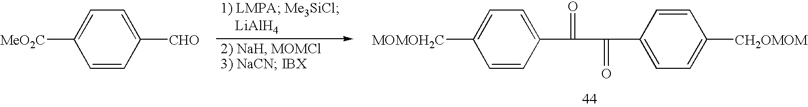

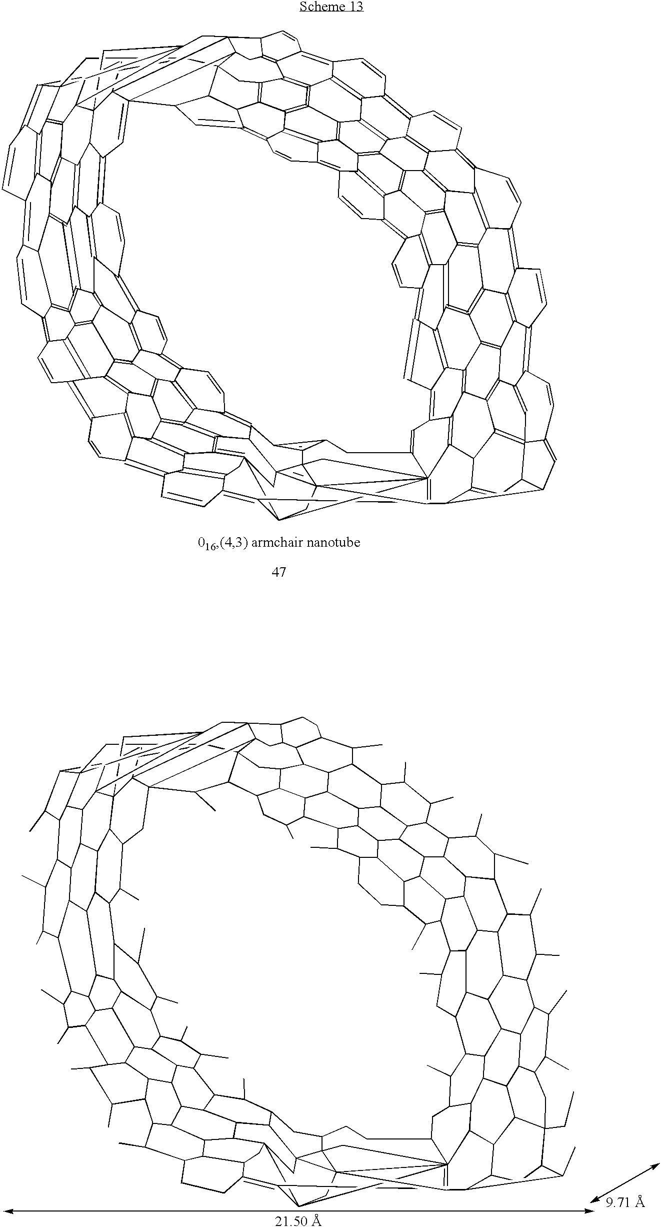

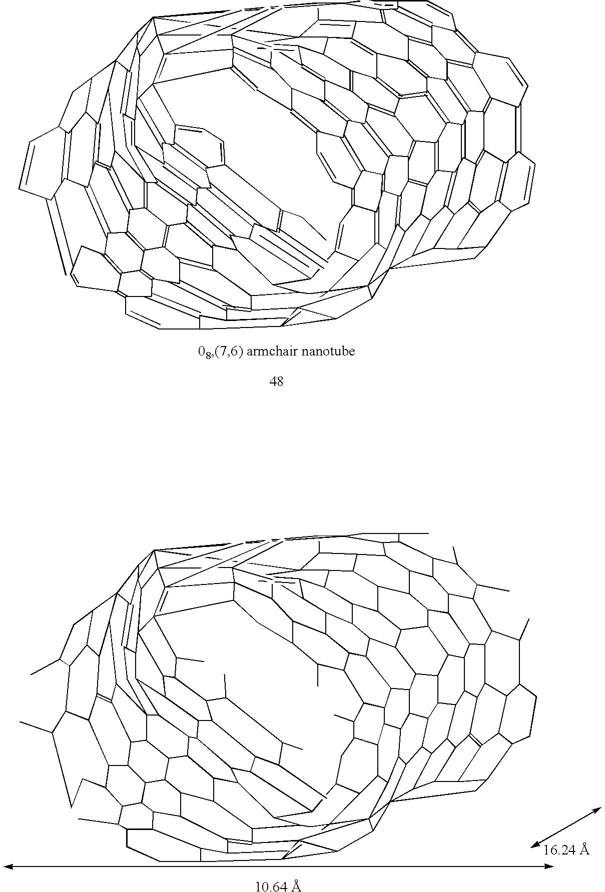

Nanotube 47 shown in Scheme 13 contains 0₁₆ benzene rings (paracyclophane) and alternating 4 and 3 stacking benzene rings, and has a diameter of 21.50 Å and a length of 9.71 Å. Nanotube 48 has a diameter of 10.64 Å and a length of 16.24 Å and contains 0₈ benzene rings (as that of nanotube 1) and alternating 7 and 6 stacking benzene rings. Compound 47 is synthesized from a symmetrical triketone 51 (Scheme 14) and is followed a similar sequence of reaction as that described for compound 1. Triketone 51 is prepared from a mono-hydrolysis of diester 15 (see Scheme 3) with 1 equivalent of potassium carbonate in methanol at 25° C. followed by bromination with triphenylphosphine and carbon tetrabromide, and carbonylation with Fe(CO)₅, Ca(OH)₂, and n-Bu₄NHSO₄ in water and dichloromethane. In the basic mono-hydrolysis of diester 15, the diol may also form, which is separated and acetylated with 1 equivalent of acetic anhydride in pyridine. Basic hydrolysis of ester 51 with excess of K₂CO₃ in methanol, silylation of the resulting diol with 2 equivalents of t-BuMe₂SiCl and triethylamine, and condensation with benzil and DBU in ethanol followed by thionyl chloride in pyridine generate tricyclopentadienone 52. Compound 52 is converted into macrocycle 53 following a similar reaction sequence as that aforementioned transformation of compound 13 to compound 8, i.e., (i) reduction of the keto function with AlCl₃-LiAlH₄, formation of triferrocenes with 6 equivalents of n-BuLi and 3 equivalents of anhydrous ferrous chloride; (iii) removal of the silyl ether protecting group and bromination with triphenylphosphine and carbon tetrabromide; (iv) macrocyclization of the triferrocenyl dibromide with Fe(CO)₅, Ca(OH)₂, and n-Bu₄NHSO₄ in water and CH₂Cl₂; (v) removal of the irons of the ferrocene moieties with lithium in n-propylamine; (vi) oxidation of the cyclopentadiene moieties with p-nitroso-dimethylaniline followed by oxidation of the hydroxyl function with IBX in DMSO; and (vii) condensation with 2 equivalents of benzil and DBU followed by dehydration with thionyl chloride in pyridine. Compound 53 is similarly converted into nanotube 47 by the Diels-Alder reactions with 8 equivalents of diphenylacetylene in refluxing diphenyl ether followed by oxidative cyclodehydrogenation with ferric trichloride in nitromethane and dichloromethane.

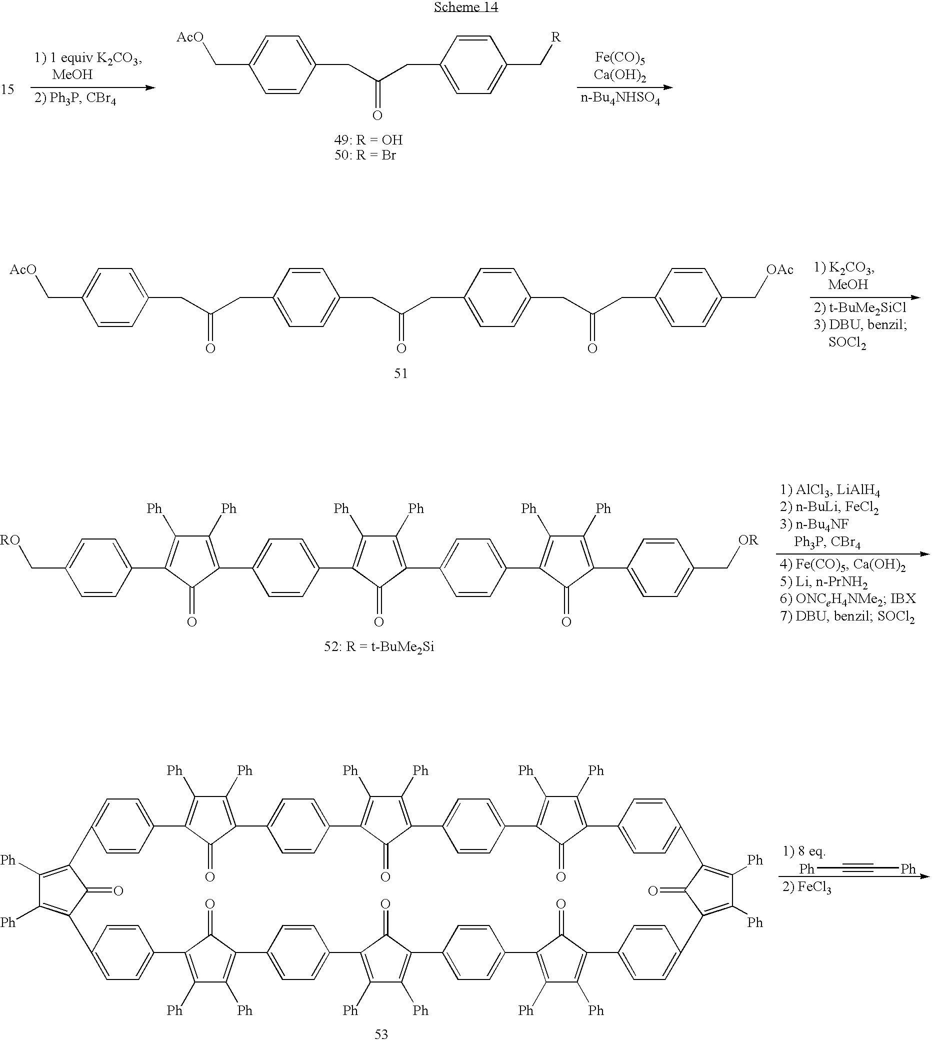

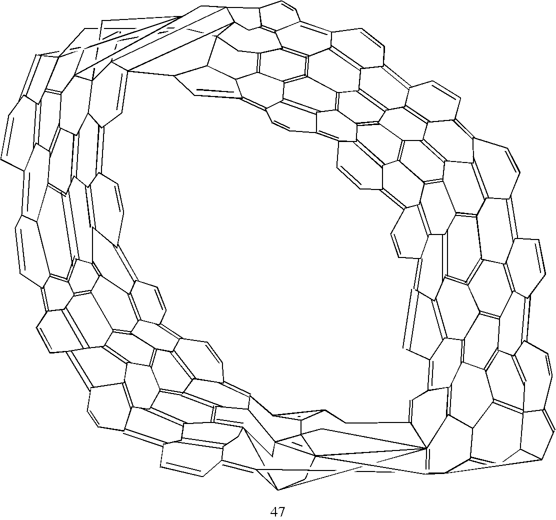

The extension of the length of the nanotube requires a slight modification of the synthesis, and the synthesis and concept are depicted in Scheme 15 and 16, respectively. The synthesis requires 1-(4-phenyl)phenyl-2-(3-phenyl)phenylethanedione (54) and 1-(4-phenylphenyl)-2-(3-phenyl)phenylethyne (57). Compound 54 is prepared from the addition reaction of Grignard reagent of bromide 58⁴⁷ with aldehyde 59⁴⁸ followed by oxidation of the resulting alcohol with IBX in DMSO, α-oxidation of the keto function with LDA and bis(trimethylsilyl)peroxide,³⁵ and oxidation with IBX in DMSO. Acetylene 57 is prepared from a similar addition reaction of Grignard reagent of bromide 58 and aldehyde 59 followed by dehydration with catalytic amounts of p-toluenesulfonic acid (p-TsOH) in toluene, bromination with bromine in CH₂Cl₂, and dehydrobromination with 4 equivalents of KOH in t-butanol.

Following a similar reaction sequence as that described above for the conversion of ketone 17 to ferrocene 11 (see Scheme 3), compound 17 is treated with benzil 54 and DBU followed by dehydration with thionyl chloride and pyridine, reduction with aluminum hydride, and formation of ferrocene with n-butyllithium and 0.5 equivalents of anhydrous ferrous chloride. Ferrocene 55 is transformed to macrocycle 56 by a similar ring closing reaction utilizing ferrocenyl moiety as an anchor: (i) removal of the silyl ether protecting group with HF or n-Bu₄NF followed by bromination; (ii) macrocyclization of the resulting dibromide with Fe(CO)₅, Ca(OH)₂, and n-Bu₄NHSO₄ in CH₂Cl₂ and H₂O; (iii) removal of the iron with lithium in n-propylamine; (iv) oxidation of the resulting cyclopentadiene moieties with p-nitroso-dimethylaniline followed by oxidation of the diol with IBX in DMSO; and (v) condensation with benzil 54 and DBU followed by dehydration with thionyl chloride in pyridine.

In the condensation reaction of the macrocycle with benzil 54, it is possible to have two other regioisomers beside 56 in which each of the two aryl moieties of the newly formed cyclopentadienones can be oriented to different directions instead of the alternating p- and m-phenyl substitutions. These regioisomers are separated by column chromatography or other methods known in the art. However, from Chem3D molecular mechanics computation, the most stable isomer is the alternating compound 56, while the two non-alternating materials are less stable and have greater repulsive forces between m-substituted phenyl rings. As indicated in the planar drawing of 60 in Scheme 16, only the alternating p- and m-phenyl substitutions provide the cyclized nanotube, 48, while other isomers do not form a nanotube. Diels-Alder reaction of compound 56 with 4 equivalents of diarylacetylene 57 in refluxing diphenyl ether followed by oxidative cyclodehydrogenation with ferric chloride in nitromethane and dichloromethane produce nanotube 48. In the Diels-Alder reaction, the predominant product is the alternating paracyclophane 60. In Scheme 16, simplified planar structures of macrocycle 56 and paracyclophane 60 are drawn for an easy view of the structures. The macrocyclic alternating cyclopentadienones and phenyls are labeled with numbers 1-8, and only rings 1-5 are depicted. When diarylacetylenes 57 approach cyclopentadienone moieties, the alternating p- and m-substitutions provide the least repulsive conformer (the most stable isomer), while other regioisomers would have a greater repulsion between the p- and m-substituted phenyl rings. The non-alternating p- and m-substituted isomers have a greater repulsion among the phenyl rings. The alternation pattern of the bottom two layers of 60 can also orient in an opposite direction, such as p- and m—(from left to right) instead of m- and p—as drawn in compound 60. Such an isomer also cyclizes to give nanotube 48.

The synthesis of nanotubes 47 and 48 involves a similar methodology to that in the synthesis of nanotube 1. The synthesis of 47 does not require the formation of three ferrocenyl rings (from compound 52), since the presence of one or two ferrocenyl rings is sufficient to facilitate the cyclization. The synthesis of nanotube 48 requires alternating p- and m-substitutions, compound 56, for the formation of the nanotube. And, compound 56 is the most stable isomer among other possible isomers, hence, it is likely to be the predominant product. The methods are general and a larger diameter and longer length of tubes such as 0₁₆; (7,6)-armchair nanotube can be synthesized by one of ordinary skill in the art using the methods described herein without undue experimentation. Functionalized derivatives of 47 and 48 can also be synthesized by one of ordinary skill in the art without undue experimentation by following similar protocols to those described herein.

### Self-Assembly of Nanotubes:

A few examples are presented here to illustrate the self-assembly of the synthesized nanotubes. First, nanotube 3 forms a stable self-assembled nanotube 61 as depicted in Scheme 17. The hydrogen of the carboxylic acid functions of compound 3 forms a hydrogen bond with two nitrogens of the bipyridyl functions of another molecule 3. Such stacking of 3 provides a long nanotube with four hydrogen bonds at each end of the small tube. Hence, a total of eight hydrogen bonds are expected from both ends of each compound, which is equivalent to ˜40 kcal/mol of interactive energy per molecule (the hydrogen bond energies vary when varying donor and acceptor groups,⁴⁹ however, an energy of ˜5 kcal/mol per hydrogen bond is typical).

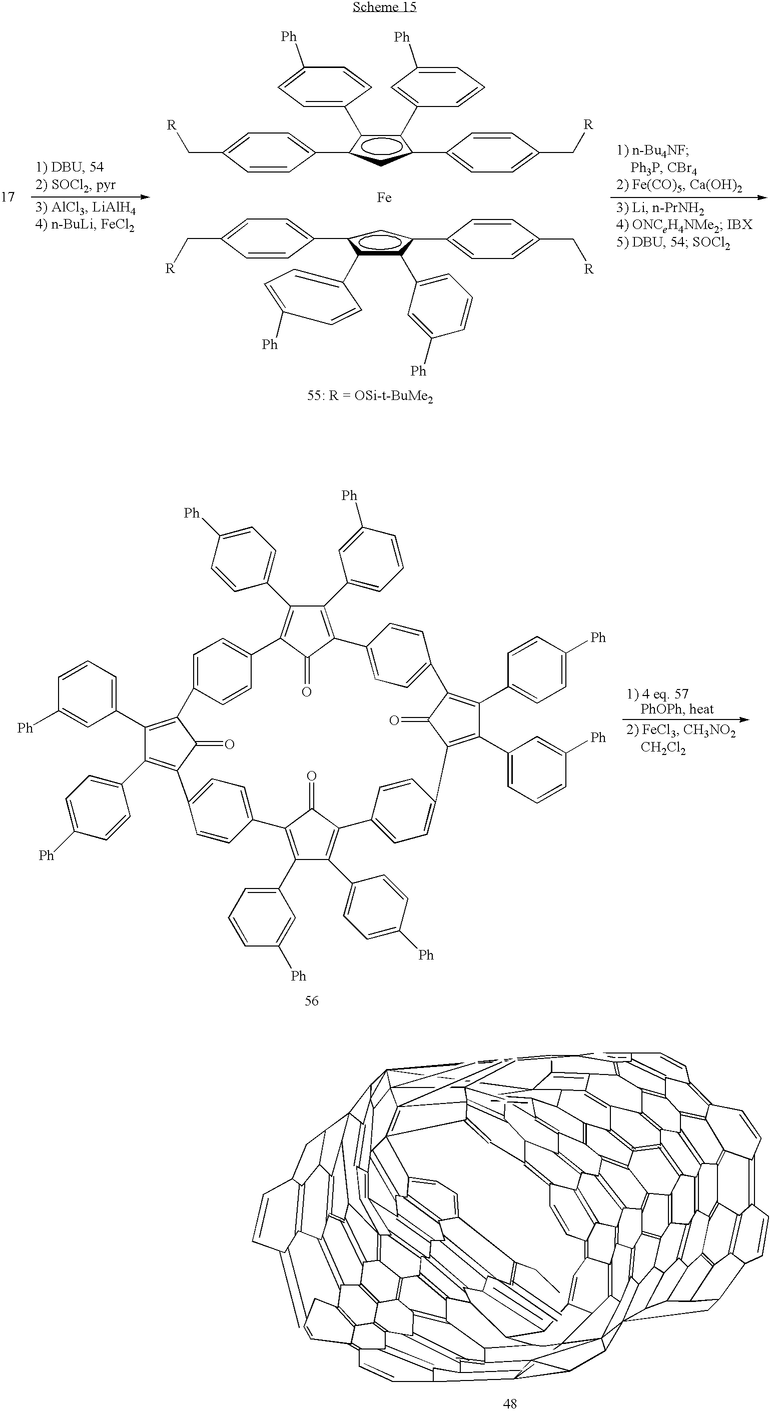

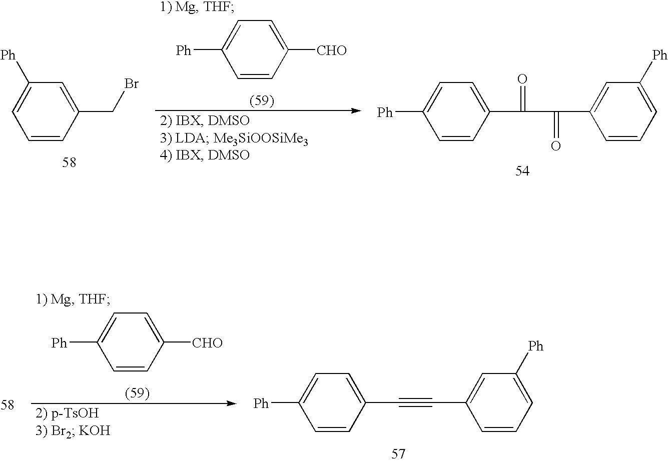

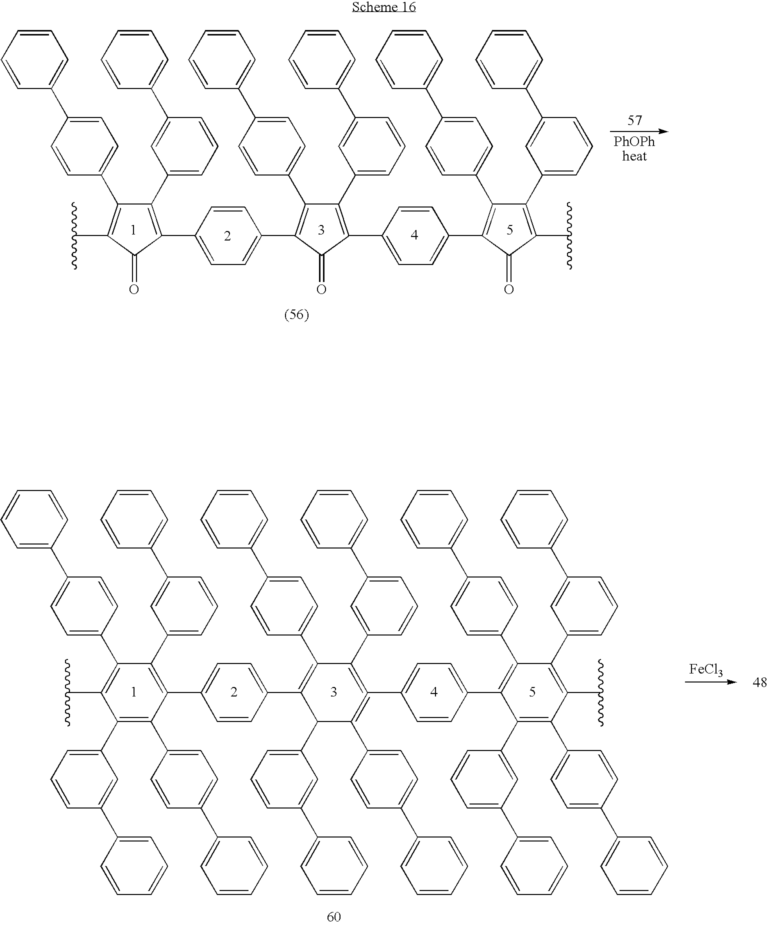

Studies on hydrogen bonding between 4,4′-bipyridyl and carboxylic acids have been reported,⁵⁰ but not between 2,2′-bipyridyl. A self-assembly from hydrogen bonds of OH groups is described next. A symmetric structure containing eight OH groups on each end, such as compound 63, is synthesized from the Diels-Alder reaction of macrocycle 38 (see Scheme 10) with 4 equivalents of diarylacetylene 62 followed by cyclodehydrogenation with ferric chloride. Self-assembled nanotube, 64, is formed from eight hydrogen bonds at one end of one molecule to one end of another molecule. The interactive force of 64 is likely to be weaker than that of 61, and their interactive energies can be measured by infrared (IR) spectroscopy from their complexation constants, hydrogen bond enthalpies, and frequency shifts.⁵¹

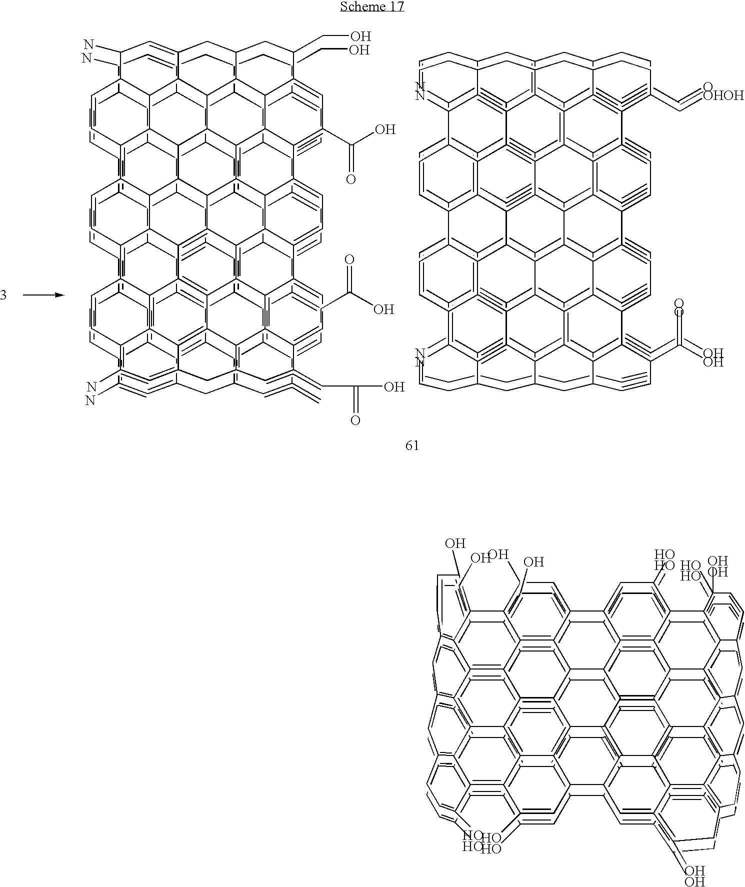

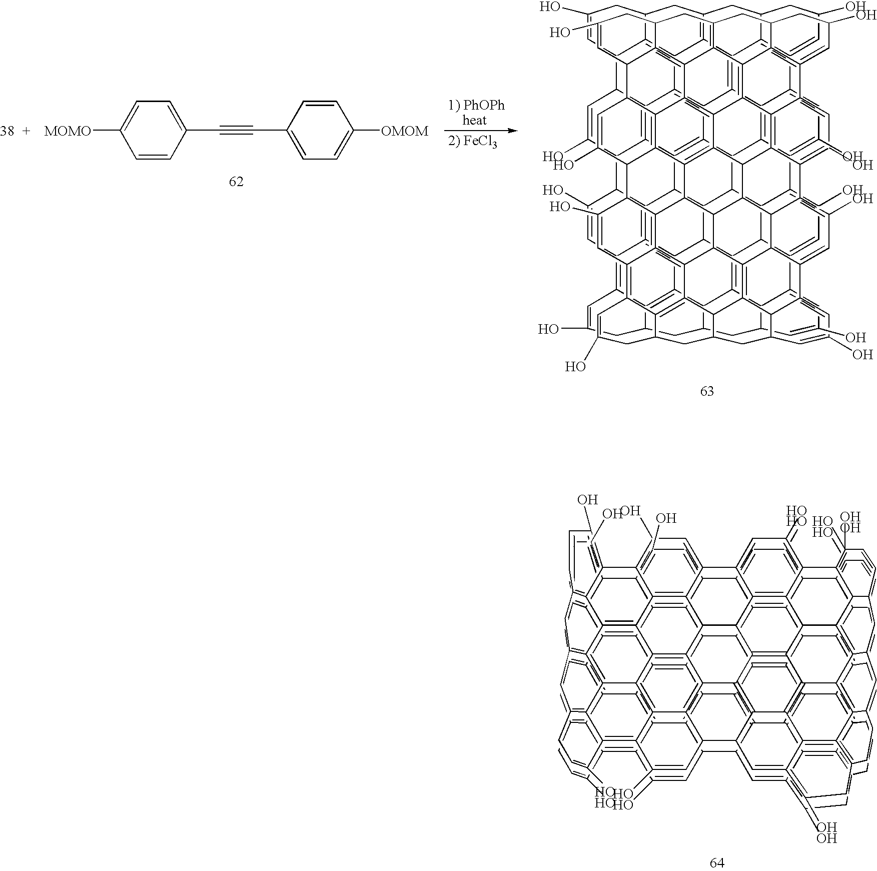

### Application of Nanotubes in Biological Systems.

Two possible applications are described here to illustrate the versatility of the synthesized carbon nanotubes: (i) formation of lipid-bilayer ion channels; and (ii) studies of peptide structures via the attachment of nanotubes onto the tip of atomic force microscopy (AFM) cantilever. As will be apparent to one of ordinary skill in the art, there are other uses of the synthesized carbon nanotubes described here.

### (i) Formation of Lipid-Bilayer Ion Channels:

As mentioned elsewhere, SWNTs have been studied as channel blockers,¹⁷ because the SWNTs are capped tubes and have an average length of ˜1 μm. The longer the tube, the more difficult the passage of chemicals through the nanotube is expected. So far, carbon nanotubes have not been reported for use in ion channels. This is not surprising since an electrostatic “dielectric barrier”⁵² is present for transferring an ion from a high dielectric phase, such as water, through a low dielectric phase, such as carbon nanotube (Born energy).⁵³ However, several computational studies have appeared recently^{52,54,55} in which water and ions such as Na⁺ are expected to pass through carbon nanotubes with a length of ˜0.8 nm and radius of ˜1.0 nm. In particular, computational studies of a (16,16) uncapped tube containing functional groups such as ammonium at one end and carboxylate anion at the other shows Cl⁻ and K⁺ ions can pass through the tube. The average occupancy of Cl⁻ ions in the tube is higher than that of the K⁺ions with a ratio of 3:2. The authors suggested⁵⁴ that the difference in water structure around the two ions along with the van der Waals interaction between the ions and the nanotube contribute to the difference of occupancies. The solvation of K⁺ion is more favorable in water than in the nanotube. Hence, studies of the passage of different ions through nanotubes having different diameters and lengths would provide experimental data to compare with that from computational studies. Undoubtedly, ions would have a greater chance of passing through nanotubes with a shorter length, such as 1 nm or less. The synthesized nanotubes described herein, in particular for example compounds 2, 3, 33-35, 47, and 48, are useful for these studies.

Cystic fibrosis transmembrane conductance regulator (CFTR), a cAMP-activated chloride (Cl⁻) channel, is located in the apical plasma membrane region in various epithelial cells and is defective in the genetic disease cystic fibrosis.^{56,57} Although synthetic Cl⁻ channel-forming peptide has been investigated to increase Cl⁻ secretion,⁵⁸ the study of nanotubes in Cl⁻ channel formation has not been reported. Nitrogen-containing nanotubes 2 and 3 may provide a pathway for the secretion of Cl⁻ in the lungs. In physiological conditions, the positively charged nitrogens of nanotubes 2 and 3 would attract negative ions such as Cl⁻ (Scheme 18).⁵⁴ The attraction would result in the movement of Cl⁻ from a higher concentration (inside of the cells) to a lower concentration (outside of the cells) in cystic fibrosis. If the bipyridyl ring systems of 2 and 3 are not the best candidates, amino-nanotube 34 (see Scheme 9) and its analog containing carboxylic acid functions at the other end of the tube are candidates for this treatment. Synthetic carbon nanotubes of this invention can be used for treatment of various problems related to chloride channels, including cystic fibrosis. This treatment involves administering an effective amount of a synthetic carbon nanotube which is effective at passing chloride ions to a patient. The synthetic carbon nanotube can be administered in a suitable carrier, as known in the art.

Potassium (K⁺) ion channels are membrane-bound macromolecules carrying out regulatory functions in almost all cell types.⁵⁹ K⁺ channels are involved in regulation of action potentials and intercellular signaling in electrically active cells, and provide a number of functions in excitable and non-excitable cells. These functions can be regulation of membrane potential and vascular tone, signal transduction, insulin secretion, hormone release, cell volume and immune response.⁵⁹ Various human diseases are related to defective K⁺ channels, which may provide a target for drug development.⁶⁰ Synthetic carbon nanotubes of this invention can be used for treatment of various problems related to defective potassium channels. This treatment involves administering an effective amount of a synthetic carbon nanotube, which is effective at passing potassium ions to a patient. The synthetic carbon nanotube can be administered in a suitable carrier, as known in the art.

Deamer and Branton has summarized an excellent account⁶¹ of the characterization of nucleic acids (such as single-stranded DNA and double-stranded DNA) using nanopores derived from proteins, such as hemolysin (with a diameter of 1.5-2.6 nm), attached to lipid bilayers. Applied voltage transports an ionic current of KCl through the open pore. The standing electrical field drives nucleic acids (ionic polymers) into the pore, consequently the current drops. The duration of the drop of current provides the length of the nucleic acid. Only single-stranded DNA passes the pore, double-stranded DNA do not.

To understand the attractive forces of different nanotubes with different ions and whether the ions can pass through the tube, we first study the passing of different ions, such as negative ions, Cl⁻, Br⁻, I⁻ and positive ions, Na⁺, K⁺, Ca²⁺ through different nanotubes (such as compounds 2, 3, 33-35, 47, 48, and 65; Scheme 18) using planar lipid bilayer experiments. In aqueous solution, ions such as K⁺or Cl⁻ are associated with several molecules of water. As described above, the protonated bipyridyl moieties of nanotube 2 would attract Cl⁻.(H₂O)ₓ ions. Nanotube 3 is similar to that computed bifunctional nanotube,⁵⁴ which possesses ammonium ions on one end and carboxylate anions on the other end of the tube. A simplified diagram of the formation of ion channels from carbon nanotubes is depicted in Scheme 18. Compound 65 is synthesized from Diels-Alder reaction of 45 (see Scheme 12) and 4 equivalents of diphenylacetylene in refluxing diphenyl ether followed by oxidative dehydrogenation with ferric chloride, removal of the MOM protecting with BF₃.ether-EtSH, and oxidation with PDC in DMF. Comparison of results of the passage of ions through nanotubes 1, 2, 64, and 3 show whether the functional groups facilitate the passage of ions. Nanotube containing amino functions, 34, and its bifunctional derivative, possessing four carboxylic acid groups on the opposite end of the amino function, is useful in studies of ion transport.

Bilayer Experiments: The procedure in published bilayer experiments⁶² is followed. Planar lipid bilayers⁶³ are formed by painting a solution of 1-palmitoyl-2-oleoyl-sn-glycero-3-phosphoethanolamine (POPE)/1-palmitoyl-2-oleoyl-sn-glycero-3-phosphocholine (POPC) (4:1 in decane with a concentration of 50 mg/mL) across a 100 μm aperture in a Teflon sheet bisecting a Lucite chamber. The hole is pre-painted with POPE/POPC (4:1) prior to membrane formation. Since different lipids may provide different ion permeabilities, different phospholipids are examined to ensure that a specific lipid can be used to obtain meaningful data for different cations or anions. The two compartments are called cis (cytosol) and trans (the lumen of the ER). A buffer solution is added to both compartments. The concentration of a target ion is varied in the cis compartment. A voltage is applied to the electrodes in the cis compartment against the electrode in the trans compartment that is connected to the ground. Ionic conductance upon changing ion concentrations in both compartments is studied. Nanotubes are either painted into the hole-area or added into the trans compartment. Single channel currents are amplified using a patch-clamp EPC9 (InstruTech Co., NY) and recorded on digital tape. The instrument is available at Kansas State University. Data is filtered with an eight-pole Bessel filter to 200 Hz, digitized to 1 kHz, transferred to a personal computer, and analyzed using Microcal Origin (North Hampton, Mass.) and PULSE (InstruTech Co., NY) software package. Conductance values are calculated from current histograms. The number of channels is not known, hence, the current will be “n×I”, where n is the number of pores and I is the current originating from one pore for a specific solution composition. The value of I can be estimated by changing the concentrations of the nanotube that is inserted into the lipid bilayer. Concentrations of the ions and nanotubes are varied to determine the efficiency of transporting different ions for each nanotube. Selectivities of different ions of a given nanotube are obtained from the slope of a linear plot of potential (E; x-axis) verses current (I; y-axis). A greater value of the slope indicates a greater selectivity. If a specific ion blocks the channels, the current drops.

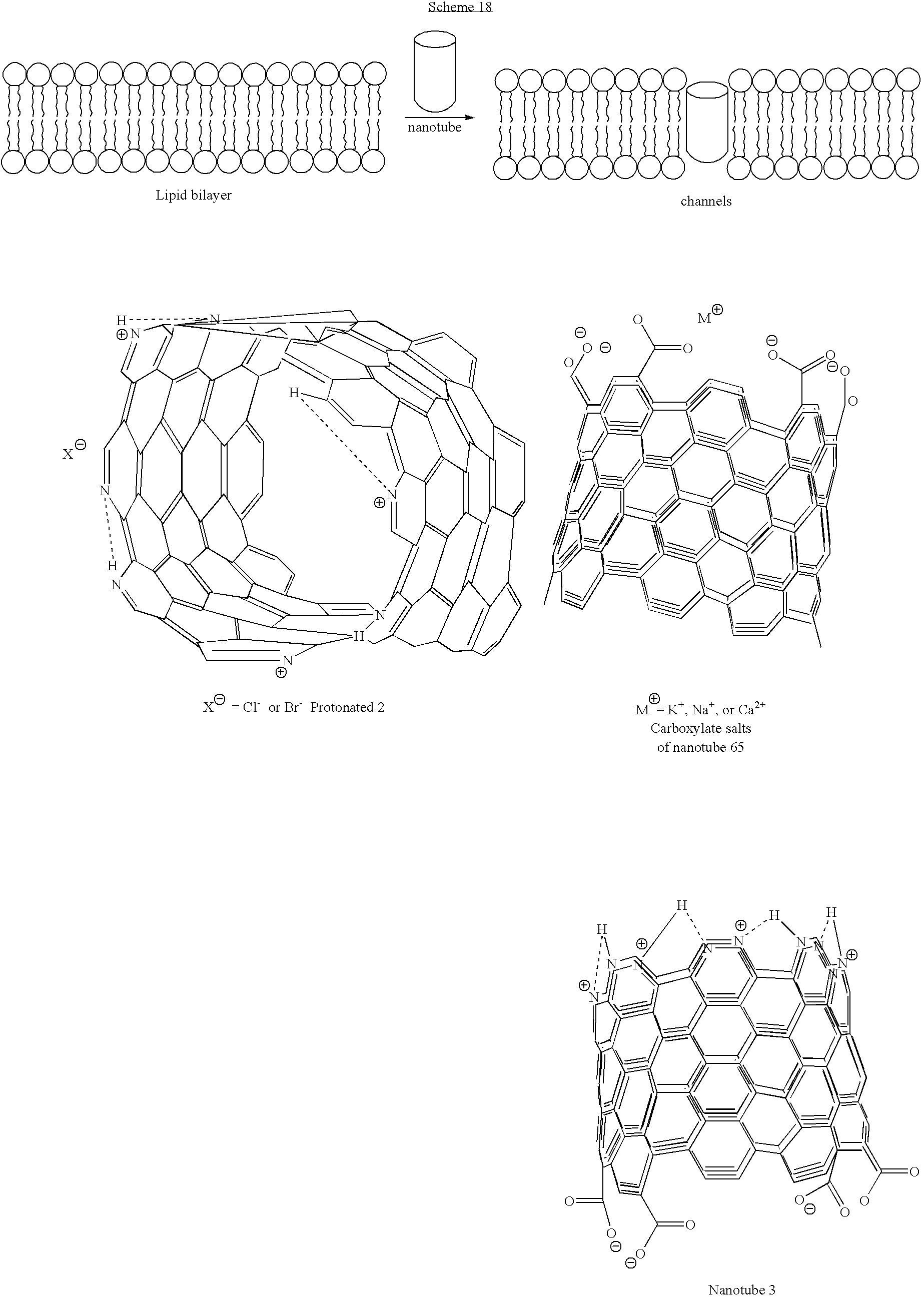

Nanotube 47, whose respective diameter and length are 2.15 nm and 0.97 nm, has a sufficient diameter for single-stranded DNA but not double-stranded DNA to pass through. Derivatives of 47 that contain amino (such as that of compound 34 or bipyridyl functions) and carboxylic acid functions similar to that of compound 3 are synthesized by following a similar protocol to that of compound 3. These synthesized carbon nanotubes are useful in characterization of nucleic acids⁶¹.

(ii) Attachment of Nanotubes onto the Tip of AFM Cantilever for Measurement of Peptides:

Currently, the resolution of AFM is ˜5 nm, which limits the use of AFM in obtaining detailed information of peptides and proteins.⁶⁴ The resolution limitation is a result of the fabrication of the tip of AFM cantilever. With the current technology, the tip of the cantilever is about 5 nm wide. Hence, an attachment of a carbon nanotube with a diameter of ˜1 nm at the tip of the cantilever would improve the resolution to 1 nm. A higher resolution of protein images was reported with an average effective radius of 3 nm^{20,65} using single-walled carbon nanotubes (SWNTs) attached AFM tips. Problems of the reported improvement of the resolution of the AFM include the difficulty in fabrication of the SWNTs onto AFM tips, the fact that various lengths and diameters of the carbon nanotubes could be formed, and the difficulty of functionalization of the AFM-tip-attached carbon nanotubes. To illustrate the application of the synthetic nanotubes described here, amyloid β peptide (Aβ) is examined. Prion proteins can similarly be studied but are not discussed here. One of ordinary skill in the art would be able to extend the discussion here to prion proteins without undue experimentation.

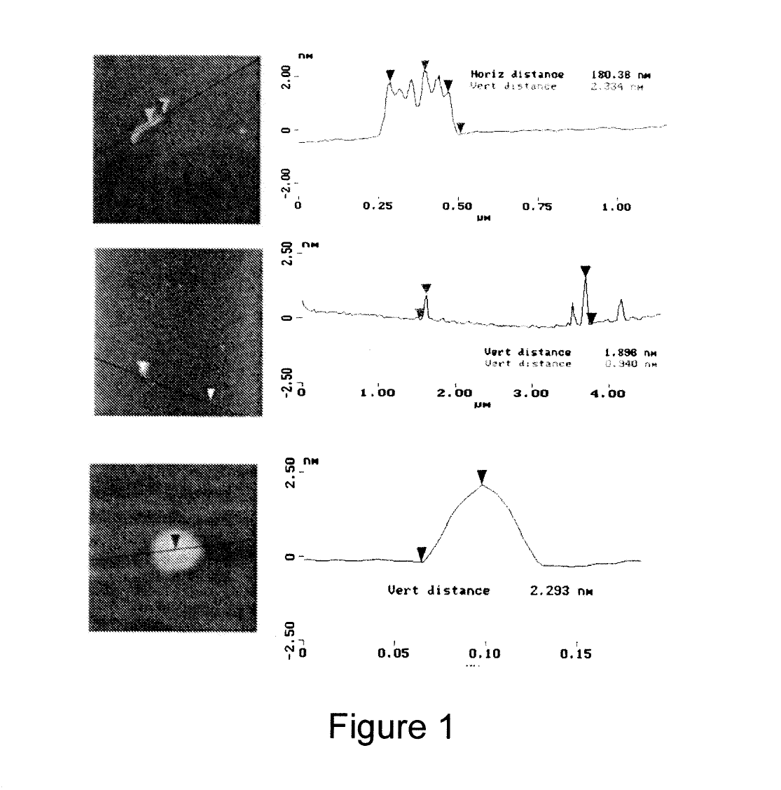

Amyloid β peptide (Aβ), a small peptide, containing 39-43 amino acids, is widely considered a culprit for Alzheimer's disease (AD). Recent evidence indicates that soluble oligomers of Aβ may represent the primary toxic species of amyloid in AD.⁶⁶ The main alloforms of Aβ deposits in AD brain are 40 and 42 amino acids long (designated as Aβ40 and Aβ42). Despite the small difference between Aβ40 and Aβ42, Aβ42 has greater neurotoxicity and forms fibril much faster than Aβ40. The secreted concentration of Aβ42 is about 10% of that of Aβ40 in a normal brain, and an increase of the Aβ42/Aβ40 concentration ratio is found in early onset of familial AD.⁶⁷ Batin et al.⁶⁸ has used DMSO (dimethyl sulfoxide) as a solvent⁶⁹ and size-exclusion chromatography to obtain pentamer and hexamer (paranuclei) of Aβ42, and electron microscopy to study the oligomers. It was suggested that these paranuclei (2-6 nm in size) aggregated to form large oligomers (20-60 nm in size), then to protofibrils (>100 nm), and to fibrils (insoluble deposits). On the other hand, Aβ40 under similar conditions assembles dimer, trimer, and tetramer, and consequently form large oligomers with a slower rate.⁶⁸ Presently, the detailed quasicircular structures⁶⁸ of Aβ42 pentamer and hexamer remain unknown. Understanding the structures of the individual monomers that made up the pentamer and hexamer provides information about the mechanism of the self-aggregation into certain shapes, which determine the rate of forming protofibril and subsequently to Aβ fibril deposits. The monomers can be α-helix, random coil, or/and β-sheet.⁷⁰ Aβ42 protofibril (derived from pH 7.4 phosphate buffer solution)⁶⁴ and small oligomers have been obtained using Batin's method,⁶⁸ and their AFM images are recorded (see FIG. 1). The protofibrils and oligomers appeared as the light areas on the Figures. The height and length of the protofibril (FIG. 1, upper panel) are 2.3 nm and 180 nm, respectively, while the height of the small oligomer is about 2.3 nm (FIG. 1, middle panel). An expansion of a small oligomer is shown in the lower panel of FIG. 1. The height is shown by the difference of two light arrows, and the width is by two dark arrows for the protofibrils. The images do not provide the shape of the small oligomers (such as a pentagon structure derived from pentamers) and the aggregation states of amino acid residues of the peptides (such as α-helix, random coil, or/and β-sheet). The attachment of synthetic carbon nanotubes onto AFM tips may provide answers to these questions.

Initially, thiol containing carbon nanotubes such as 35 attached to AFM tips (gold tips) (Scheme 19) are used to study structures of small oligomers such as Aβ42 pentamers and hexamers, and protofibrils. The longer nanotube 70, a thiol containing 48, is then used. Compound 70 is synthesized from 55 (see Scheme 15) by following a similar reaction as that described for the synthesis of compound 48, but using benzil 72 (in place of 54) and diarylacetylene 73 (instead of 57), and displacing the bromines with EtOH(OSiMe₃)SLi, KF, and hydrolysis with KOH. A diluted solution of nanotube 35 is prepared, a gold tip of the AFM cantilever allowed to contact onto the surface of the solution, and the thiol functions of nanotube 35 link to the gold surface via sulfur-gold bonds to give 66. Similarly, compound 70 is attached to a separate gold tip.

Since structural information of proteins can be obtained from adhesive forces,²⁰ the adhesive forces between the oligomers and protofibrils are measured with different groups attached to the open end of nanotubes by following the reported procedure^{71,72} in the modification of SWNTs tips. Hence, thiol 67 is attached to a cantilever gold tip, and the amino functions are condensed with various carboxylic acids using the N-hydroxysuccinimide activation protocol. Aminothiol nanotube 67 is synthesized from ketone 9 (see Scheme 12) with benzil 74 and DBU, followed by thionyl chloride in pyridine, Diels-Alder reaction with 4 equivalents of diarylacetylene 26, cycldehydrogenation with FeCl₃, displacement of the bromine moieties with EtOH(OSiMe₃)SLi, KF, and basic hydrolysis of the amide functions with KOH. After attachment of 67 onto the gold tip, the amino functions are condensed with imide 68 to give various functionalized tubes 69. Imide 68 is prepared from various carboxylic acids (the amino group is protected with two Boc groups⁷³) and N-hydroxysuccinimide and N,N′-dicyclohexylcarbodiimide. The carboxylic acids can be phenylacetic acid or bis-Boc-NCH₂CH₂CH₂CO₂H.⁷³ Removal of the Boc protecting groups with trifluoroacetic acid after the amide formation provide ammonium salt 69B. These modified carbon nanotube tips are used to study the adhesive forces²⁰ between functionalized nanotube tips and Aβ42 pentamer and hexamer, and protofibrils. The benzyl amide tip detects the hydrophobic interaction areas of Aβ42 such as the fragment containing residues Gly(29) to Ala(42) (β-sheet fragment).⁷² The ammonium propyl amide tip 69B at neutral pH provides stronger interactions with Asp(1) to Glu(3) fragment (ionic attractive force) and hydrophilic fragments.⁷⁴ The adhesion data provide the interactive areas between monomers and possible interactive sites of Aβ42. Similarly, longer nanotubes 71A and 71B are used for the attachment to gold tips and the studies of the interactive sites of oligomers.

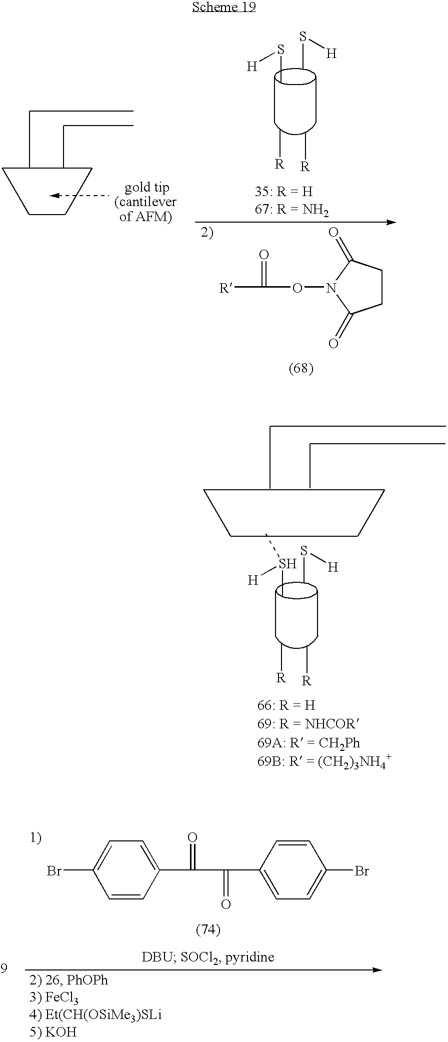

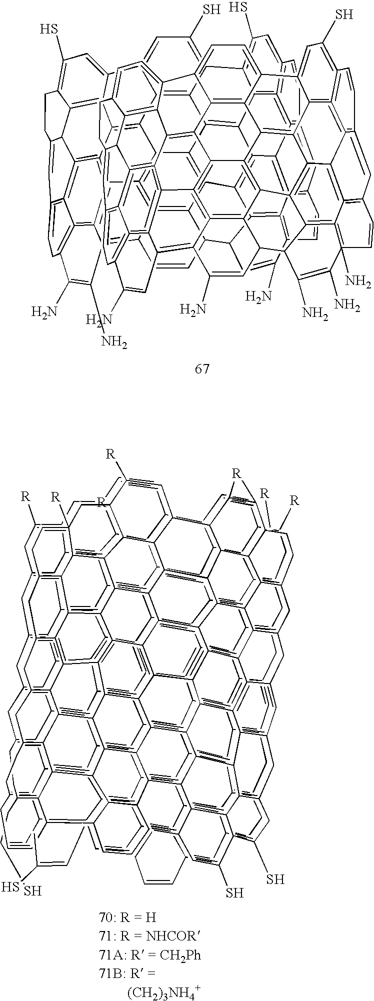

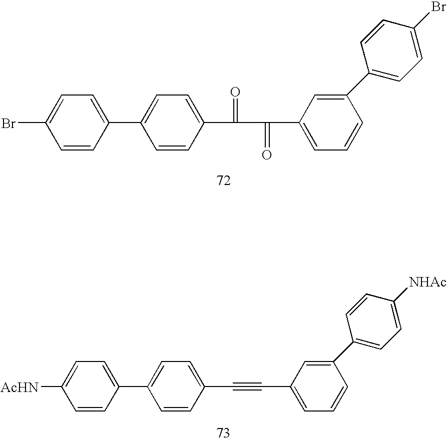

### Experimental Section

General Methods. Unless otherwise indicated, NMR spectra were obtained at 400 MHz for ¹H and 100 MHz for ¹³C in CDCl₃, and reported in ppm. Infrared spectra are reported in wavenumbers (cm⁻¹). Mass spectra were taken from a Bruker Esquire 3000 Plus electrospray ionization mass spectrometer and a MALDI-TOF/TOF MS instrument, Model; Ultraflex II (Bruker Daltonics). High-resolution Mass spectra were taken from an IonSpec HiResMALDI mass spectrometer using 2,5-dihydroxybenzoic acid as a matrix. Silica gel, grade 643 (200˜425 mesh), was used for the flash column chromatographic separation. Tetrahydrofuran and diethyl ether were distilled over sodium and benzophenone before use. Methylene chloride was distilled over CaH₂ and toluene and benzene were distilled over LiAlH₄. FeCl₂ was purchased from Strem Chemical Company. Other chemicals and reagents were purchased either from Aldrich Chemical Company or Fisher Chemical Company, and were used without purification.

Preparation of 1,4-di(bromomethyl)benzene. A solution of 1.23 mL (10 mmol) of p-xylene, 3.54 g (20 mmol) of N-bromosuccinimide (NBS) and 45 mg (0.2 mmol) of benzoyl peroxide in 30 mL of benzene was heated under reflux (80° C.) and argon atmosphere for 4 hours. The mixture was cooled to 25° C. and diluted with aqueous NH₄OH and NaHCO₃, and solids (succinimide) were removed by filtration. The filtrate was extracted twice with diethyl ether, and the combined ether extract was washed with brine, dried (anhydrous Na₂SO₄), and concentrated to give white solids. The solids were crystallized in diethyl ether to provide 1.20 g (45.5% yield) pure product, 1,4-di(bromomethyl)benzene, as white solids. The mother liquor was concentrated to give 1.40 g of a mixture of the product and by-products. ¹H NMR 7.37 (s, 4H, Ar), 4.48 (s, 4H, CH₂); ¹³C NMR 138.2 (s), 129.7 (d), 33.0 (t).

Preparation of 4-(bromomethyl)benzyl acetate (14). A solution of 13.4 g (50.6 mmol) of 1,4-di(bromomethyl)benzene and 4.96 g (50.6 mmol) of potassium acetate in 80 mL of acetonitrile (HPLC grade) was stirred for 16 h under argon, and acetonitrile was removed using a rotary evaporator. The crude product was dissolved in 100 mL of ethyl acetate and washed with water, aqueous NH₄Cl, and brine. The organic layer was dried (anhydrous Na₂SO₄), concentrated, and the resulting semi-solid liquid was diluted with 30 mL of hexane:ethyl acetate (20:1) and filtered through a fritted funnel to remove unreacted 1,4-di(bromomethyl)benzene (solid). The filtrate was concentrated and column chromatographed on silica gel using a gradient mixture of hexane and ethyl acetate to give 7.15 g (58% yield). The above solid gave 1.5 g (11.2% recovery) of starting material. ¹H NMR 7.39 (d, J=8 Hz, 2H, Ar), 7.34 (d, J=8 Hz, 2H, Ar), 5.10 (s, 2H, CH₂OAc), 4.49 (s, 2H, CH₂Br), 2.11 (s, 3H, CH₃);28 ¹³C NMR 170.4 (s, CO), 141.6 (s), 137.6 (s), 127.9 (d), 126.4 (d), 64.9 (t, CH₂O), 59.9 (t, CH₂Br), 13.9 (q).

1,3-Di[(4-acetoxymethyl)phenyl]propanone (15). To a dry flask, 1.40 g (19 mmol) of Ca(OH)₂ and 0.80 g (2.4 mmol) of n-Bu₄NHSO₄ were added. The materials were vacuum and flame dried, and maintained under argon. To the mixture, 100 mL of degassed water and dichloromethane (1:1) were added followed by the additions of 2.30 g (9.5 mmol) of 4-(bromomethyl)benzyl acetate (14) and 0.93 g (4.73 mmol) of Fe(CO)₅ (freshly distilled) via syringe. After stirring at 25° C. for 5 h, the reaction solution was aerated by bubbling air in for 30 min to oxidize unreacted irons. The mixture was filtered through a fritted funnel and washed with ethyl acetate. The filtrate was washed with aqueous NH₄Cl, water, and brine, dried (MgSO₄), concentrated, and column chromatographed on silica gel using a gradient mixture of hexane and ethyl acetate as eluants to give 1.28 g (76% yield) of compound 15. ¹H NMR 7.32 (d, J=8 Hz, 4H, Ar), 7.16 (d, J=8 Hz, 4H, Ar), 5.09 (s, 4H, CH₂OAc), 3.73 (s, 4H, CH₂CO), 2.11 (s, 6H, CH₃); ¹³C NMR 207.2 (s, CO), 171.5 (s, CO), 135.0 (s), 134.1 (s) 129.9 (d), 128.9 (d), 66.1 (t, CH₂O), 49.0 (t, CH₂), 21.2 (q).

1,3-Di[(4-hydroxymethyl)phenyl]propanone (16). A solution of 0.79 g (2.22 mmol) of diacetate 15 and 1.22 g (8.87 mmol) of potassium carbonate (anhydrous) in 15 mL of methanol (distilled over Mg turning) was stirred under argon at 25° C. for 3 h. The reaction solution was diluted with ethyl acetate and neutralized carefully with 1 N HCl, and the organic layer was washed with brine, dried (MgSO₄), and concentrated to give 0.542 g (91% yield) of compound 16, which was used in the next reaction without further purification. ¹H NMR 7.32 (d, J=8 Hz, 4H, Ar), 7.15 (d, J=8 Hz, 4 H, Ar), 4.69 (s, 4H, CH₂OH), 3.73 (s, 4H, CH₂CO), 1.59 (bs, 2H, OH); ¹³C NMR 206.2 (s, CO), 139.9 (s), 133.3 (s), 129.8 (d), 127.6 (d), 64.9 (t, CH₂O), 49.0 (t, CH₂).

1,3-Bis{[4-t-butyldimethylsilyloxy)methyl]phenyl}propanone (17). A solution of 0.54 g (2.01 mmol) of diol 16, 0.61 g (6.03 mmol) of triethylamine (distilled over CaH₂), 50 mg (0.40 mmol) of 4-(dimethylamino)pyridine (DMAP), and 1.21 g (8.03 mmol) of t-butyldimethylsilyl chloride in 15 mL of dichloromethane was stirred under argon at 25° C. for 24 h. The solution was diluted with diethyl ether, washed with aqueous NH₄Cl, water, and brine, dried (anhydrous Na₂SO₄), concentrated, and column chromatographed on silica gel using a gradient mixture of hexane and diethyl ether to give 0.92 g (92% yield) of compound 17. ¹H NMR 7.27 (d, J=8 Hz, 4H, Ar), 7.11 (d, J=8 Hz, 4H, Ar), 4.73 (s, 4H, CH₂OSi), 3.69 (s, 4H, CH₂CO), 0.94 (s, 18H, t-Bu); 0.10 (s, 12H, MeSi); ¹³C NMR 206.0 (s, C═O), 140.5 (s), 132.8 (s), 129.5 (d), 126.6 (d), 64.9 (t, CH₂O), 48.9 (t, CH₂), 26.2 (q, 6 C), 18.6 (s, CSi), −5.1 (q, 4 C).

2,4-Bis{[(4-t-butyldimethylsilyloxy)methyl]phenyl}-1,5-diphenyl-3-oxo-4-cyclopentenol (18). A solution of 2.18 g (4.38 mmol) of ketone 17 and 0.67 g (4.38 mmol) of 1,8-diazabicyclo[5.4.0]undec-7-ene (DBU) in 30 mL of ethanol (distilled over Mg turning) was stirred for 30 min under argon. To it, a solution of 0.92 g (4.38 mmol) of benzil in 10 mL of ethanol was added via a cannula, and the solution was stirred at 25° C. for 1 day and 45° C. for 2 h. Ethanol was removed using a rotary evaporator and the residue crude product was used in the next transformation without further purification.

2,5-Bis{[(4-t-butyldimethylsilyloxy)methyl]phenyl}-3,4-diphenyl-2,4-cyclopentadienone (13). To the above crude product 18 under argon at 0° C., 10 mL of pyridine and 2 mL of thionyl chloride were added, and the solution was stirred at 0° C. for 25 min. The solution was diluted with diethyl ether, washed with aqueous NH₄Cl and brine, dried (anhydrous Na₂SO₄), concentrated and column chromatographed on silica gel using a gradient mixture of hexane and diethyl ether as eluants to give 2.50 g (86% yield; 2 steps from compound 17) of 13. ¹H NMR 7.25-7.14 (m, 14H, Ar), 6.92 (d, J=7 Hz, 4H, Ar), 4.71 (s, 4H, CH₂OSi), 0.93 (s, 18H), 0.08 (s, 6H), 0.075 (s, 6H); ¹³C NMR 208.0 (s, C═O), 154.4 (s), 140.8 (s), 135.1 (s), 133.4 (s), 130.2 (d), 130.1 (d), 129.6 (d), 129.2 (d), 128.6 (d), 128.2 (d), 125.9 (d), 125.4 (s), 65.0 (t, CH₂O), 26.2 (q), 18.6 (s), −5.0 (q).

1,4-Bis{[(4-t-butyldimethylsilyloxy)methyl]phenyl}-2,3-diphenyl-1,3-cyclopentadiene (19). To a solution of 0.15 g (0.22 mmol) of ketone 13 in 2.5 mL of diethyl ether under argon at 0° C., were added 15 mg (0.11 mmol) of AlCl₃ followed by 4 mg (0.11 mmol) of LiAlH₄. The solution turned purple color to yellowish green upon addition of LiAlH₄. After the additions, the reaction mixture was heated to reflux for 5 h, cooled to 25° C., added carefully water to destroy excess of LiAlH₄, diluted with diethyl ether, washed with water and brine, dried (anhydrous Na₂SO₄), concentrated, and column chromatographed on silica gel using a gradient mixture of hexane and ether as eluants to give 79 mg (55% yield) of compound 19. MS (electrospray ionization) m/z 543.40 (M-t-BuMe₂Si-1); ¹H NMR 7.15 (m, 14H), 6.97 (m, 4H), 4.68 (s, 4H, CH₂O), 4.01 (s, 2H, CH₂), 0.92 (s, 18H), 0.08 (s, 12H); ¹³C NMR 144.3, 139.5, 136.7, 135.2, 129.9, 128.3, 126.0, 127.7, 126.6, 125.9, 64.8, 45.9, 26.0, −5.24.

1,4,1′,4′-Tetra[4-(t-butyldimethylsilyloxy)methylphenyl]-2,3,2′,3′-tetraphenylferrocene (11). A two-necked round-bottom flask was equipped with a solid addition tube in one of the necks. To it were added 0.30 g (0.46 mmol) of cyclopentadiene 19 to the flask and 58 mg (0.46 mmol) of FeCl₂ (anhydrous) to a flask attached to the solid addition tube under argon. The apparatus was dried under vacuum and heat, and 2 mL of THF was added via syringe to the flask containing cyclopentadiene 19. The solution was cooled to −78° C., added 0.58 mL (0.69 mmol) of n-BuLi (1.2 M in pentane), stirred for 1.5 h, added FeCl₂ through addition tube, and stirred at 25° C. for 15 h under argon. The solution was diluted with ethyl acetate, washed with aqueous NH₄Cl and brine, dried (anhydrous Na₂SO₄), concentrated, and column chromatographed on silica gel using a gradient mixture of hexane and diethyl ether as eluants to give 0.22 g (70% yield) of compound 11. MS (electrospray ionization) m/z 1371.40 (M+1), 1370.60 (Ar); exact mass calc. for C₈₆H₁₀₆FeO₄Si₄+H⁺: 1372.65 (M+1); ¹H NMR 7.05 (d, J=8 Hz, 8H), 6.96-6.90 (m, 20H), 6.83 (t, J=8 Hz, 8H), 5.49 (s, 2H, Cp), 4.62 (s, 8H, CH₂O), 0.94 (s, 36H), 0.09 (s, 24H); ¹³C NMR 139.4, 135.2, 134.1, 132.3, 129.2, 126.8, 126.1, 125.4, 91.4 (Cp), 86.0 (Cp), 67.8 (Cp), 65.0 (CH₂O), 26.0, −5.2.

1,4,1′,4′-Tetra[4-(hydroxymethyl)phenyl]-2,3,2′,3′-tetraphenylferrocene (74). To a solution of 0.15 g (0.11 mmol) of compound 11 in 1 mL of THF under argon at 0° C. was added 4.4 mL of n-Bu₄NF in THF (0.05 M). The solution was stirred at 25° C. for 4 h, diluted with dichloromethane, washed with aqueous NH₄Cl, water, and brine, dried (anhydrous Na₂SO₄), concentrated to give 0.144 g of compound 74 and t-butyldimethylsilyl alcohol. This material was used in the next step without further purification. MS (HiRes MALDI) m/z 914.2417; Calcd for C₆₂H₅₀FeO₄: 914.31 (exact mass); ¹H NMR 7.3-6.7 (m, 36H), 5.46 (s, 2H, Cp), 4.7-4.5 (m, 8H, CH₂O); ¹³C NMR (D₂O—CH₃₀D) 139.9, 136.0, 132.3, 129.4, 127.1, 126.7, 126.5, 118.8, 90.9 (Cp), 80.9 (Cp), 64.4 (CH₂O).

1,4,1′,4′-Tetra(4-formylphenyl)-2,3,2′,3′-tetraphenylferrocene (75). To a solution of 0.144 g (from the above crude product) of 74 in 10 mL of DMSO (distilled over CaH₂) under argon was added 0.22 g (0.79 mmol) of IBX. The solution was stirred at 25° C. for 3 h, diluted with water, and extracted three times with ethyl acetate. The combined extract was washed with brine, dried (anhydrous Na₂SO₄), concentrated and column chromatographed on silica gel using a gradient mixture of hexane, dichloromethane and ethyl acetate to give 0.141 g (99% yield) of tetraaldehyde 75.

¹H NMR 9.90 (s, 4H, CHO), 8.05 (d, J=8 Hz, 1H), 7.99 (d, J=8 Hz, 1H), 7.49 (d, J=8 Hz, 6H), 7.25 (d, J=8 Hz, 8H), 7.04 (m, 4H), 6.88 (m, 16H), 5.77 (s, 2H, Cp).¹³C NMR 191.8, 142.0, 141.6, 134.8, 133.4, 132.0, 131.3, 129.7, 129.3, 127.6, 104.0.

1,4,1′,4′-Tetra[4-(1-hydroxy-2-propenyl)phenyl]-2,3,2′,3′-tetraphenylferrocene (76). To a solution of 66 mg (0.07 mmol) of tetraaldehyde 75 in 5 mL of THF under argon, was added 0.44 mL (0.44 mmol) of vinylmagnesium bromide (1.0M in THF). The solution was stirred at 25° C. for 3 h, diluted with aqueous NH₄Cl, and extracted twice with ethyl acetate. The combined extract was washed with brine, dried (anhydrous Na₂SO₄), concentrated, and column chromatographed on silica gel using a gradient mixture of hexane, ethyl acetate, and methanol as eluants to give 44 mg (60% yield) of tetraol 76. HRMS (MALDI) m/z 1019.033 (M+1; 100%), 1021.037 (79%, isotope), 1022.040 (29%, isotope); Calcd for C₇₀H₅₈FeO₄: 1018.37 (M⁺); ¹H NMR 7.12-6.80 (m, 36H), 6.0 (m, 4H, CH═), 5.6-5.0 (a serious of m, 14H), 2.2 (bs, 4H, OH). ¹³C NMR 140.6, 140.4, 140.3, 140.2, 135.2, 135.0, 134.9, 134.6, 132.5, 129.3, 129.2, 127.1, 126.4, 125.8, 115.3, 115.2, 115.1, 92.2, 85.7, 75.3.

Ferrocenocyclophane 77. A solution of 33 mg (0.03 mmol) of tetraol 76 and 2.7 mg (1.6 μM) of Grubbs' 2nd generation catalyst in 8 mL of benzene under argon was stirred at 45-50° C. for 1 day. The solution was diluted with dichloromethane, washed with aqueous NH₄Cl and brine, dried (MgSO₄), concentrated to give ferrocenocyclophane 77 along with the trimer and uncyclized dimer (one olefin metathesis had taken place). Compound 77 was identified by mass spectrometry: MS (MALDI) m/z 1925.74 (M⁺), 1924.75 (M−1); exact mass Calcd for C₁₃₂ H₁₀₀Fe₂O₈: 1925.62 (100%); ¹H NMR 7.2-6.6 (m, 72H, Ar), 6.2-5.0 (m, 16H).

Ferrocenocyclophane 78. A solution of 33 mg (0.03 mmol) of tetraol 76 and 2.7 mg (1.6 μM) of Grubbs' 2nd generation catalyst in 40 mL of benzene under argon was stirred at 45-50° C. for 1 day. The solution was diluted with dichloromethane, washed with aqueous NH₄Cl and brine, dried (MgSO₄), concentrated to give ferrocenocyclophane 78 as the major product. MS (MALDI) m/z 962.219 (M⁺), 963.224 (M+1), 964.222 (M+2). ¹H NMR (CDCl₃+CH₃OD) 7.2-6.7 (m, Ar), 6.06 (s, 4H, ═CH), 5.79 (s, 2H, Cp), 4.73 (bs, 4H, CHO).

When a group of substituents is disclosed herein, it is understood that all individual members of those groups and all subgroups, including any isomers and enantiomers of the group members, and classes of compounds that can be formed using the substituents are disclosed separately. When a compound or method is claimed, it should be understood that compounds or methods known in the art including the compounds or methods disclosed with an enabling disclosure in the references disclosed herein are not intended to be included. When a Markush group or other grouping is used herein, all individual members of the group and all combinations and subcombinations possible of the group are intended to be individually included in the disclosure.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomer and enantiomer of the compound described individual or in any combination. One of ordinary skill in the art will appreciate that methods, steps, and starting materials other than those specifically exemplified can be employed in the practice of the invention without resort to undue experimentation. All art-known functional equivalents of any such methods steps and starting materials are intended to be included in this invention. Whenever a range is given in the specification, for example, a temperature range, a time range, a particle size range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

In general the terms and phrases used herein have their art-recognized meaning, which can be found by reference to standard texts, journal references and contexts known to those skilled in the art. The definitions are provided to clarify their specific use in the context of the invention.

All patents and publications mentioned in the specification are indicative of the levels of skill of those skilled in the art to which the invention pertains.

One skilled in the art would readily appreciate that the present invention is well adapted to carry out the objects and obtain the ends and advantages mentioned, as well as those inherent therein. The compounds used, products formed and methods and accessory methods described herein as presently representative of preferred embodiments are exemplary and are not intended as limitations on the scope of the invention. Changes therein and other uses will occur to those skilled in the art, which are encompassed within the spirit of the invention, are defined by the scope of the claims.

Although the description herein contains many specificities, these should not be construed as limiting the scope of the invention, but as merely providing illustrations of some of the embodiments of the invention. Thus, additional embodiments are within the scope of the invention and within the following claims. All references cited herein are hereby incorporated by reference to the extent that there is no inconsistency with the disclosure of this specification. Some references provided herein are incorporated by reference herein to provide details concerning additional starting materials, additional methods of synthesis, additional methods of analysis and additional uses of the invention.

The exact formulation, route of administration and dosage of synthetic carbon nanotubes used in the treatment of patients can be chosen by the individual physician in view of the patient's condition (see e.g. Fingl et. al., in The Pharmacological Basis of Therapeutics, 1975, Ch. 1 p. 1). Patients which can be treated include mammals. One class of mammals is humans. One class of mammals is small animals such as dogs and cats. One class of mammals is large animals such as cows, pigs and sheep.

It should be noted that the attending physician would know how to and when to terminate, interrupt, or adjust administration due to toxicity, or to organ dysfunctions, or to other adverse reactions. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above also may be used in veterinary medicine.

Depending on the specific conditions being treated and the targeting method selected, such agents may be formulated and administered systemically or locally.

Techniques for formulation and administration may be found in Alfonso and Gennaro (1995). Suitable routes may include, for example, oral, rectal, transdermal, vaginal, transmucosal, or intestinal administration; parenteral delivery, including intramuscular, subcutaneous, or intramedullary injections, as well as intrathecal, intravenous, or intraperitoneal injections.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular those formulated as solutions, may be administered parenterally, such as by intravenous injection. Appropriate compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve the intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions, including those formulated for delayed release or only to be released when the pharmaceutical reaches the small or large intestine.

The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

### REFERENCES

1. Herges, R. Fully conjugated beltenes (belt-like and tubular aromatics). In “Modern cyclophane chemistry”; Gleiter, R.; Hopf, H. Ed., Wiley-VCH Verlag GmbH & Co. KgaA, Weinheim, 2004, p 337-358.
2. Watson, M. D.; Fechtenkotter, A.; Mullen, K. Big is beautiful-“aromaticity” revisited from the viewpoint of macromolecular and supramolecular benzene chemistry. Chem. Rev. 2001, 101, 1267-1300.
3. Bong, D. T.; Clark, T. D.; Granja, J. R.; Ghadiri, M. R. Self-assembling organic nanotubes. Angew. Chem. Int. Ed. 2001, 40, 988-1011.
4. Schroder, A.; Mekelburger, H.-B.; Vogtle, F. Belt-, ball-, and tube-shaped molecules. Top. Curr. Chem. 1994, 172, 179-201.
5. Seel, C.; Vogtle, F. Molecules with large cavities in supramolecular chemistry. Angew. Chem. Int. Ed. Engl. 1992, 31, 528-49.
6. van Nostrum, C. F.; Picken, S. J.; Schouten, A.-J.; Nolte, R. J. M. Synthesis and supramolecular chemistry of novel liquid crystalline crown ether-substituted phthalocyanines. Toward molecular wires and molecular ionoelectronics. J. Am. Chem. Soc. 1995, 117, 9957-65.
7. Subramoney, S. Novel nanocarbons-structure, properties, and potential applications. Adv. Mater. 1998, 10, 1157-71.
8. Harada, A.; Li, J.; Kamachi, M. Synthesis of a tabular polymer from threaded cyclodextrins. Nature, 1993, 364, 516-8.
9. Hua, D. H.; McGill, J. W.; Lou, K.; Ueki, A.; Helfrich, B.; Desper, J.; Zanello, P. Parallel and perpendicular stacking of ferrocene rings. Synthesis, x-ray structures, and electrochemistry of 1,8-bis-[1-(1′-phenylthio)ferrocenyl]naphthalene and 8,8′-bis-[1-(1′-phenylthio)ferrocenyl]-1,1′-binaphthyl. Inorganica Chim. Acta. 2003, 350, 259-265.
10. Hua, D. H.; McGill, J. W.; Ueda, M.; Stephany, H. A. Short Syntheses of 8′-Substituted 8-[1-(1′-Phenylsulfinyl)ferrocenyl]-1,1′-binaphthyls From the Suzuki Coupling Reactions. A Strategy For Generating New Chiral Ligands and Charge-Transfer Complexes. J. Organomet. Chem. 2001, 637-639, 832-836.
11. Hua, D. H.; McGill, J. W.; Robinson, P. D.; Meyers, C. Y. 2,4,6-Tris(1′-phenylthio-1-ferrocenyl)boroxin. Acta Cyst. 2001, E57, m413-m415.
12. Lagneau, N. M.; Chen, Y.; Robben, P. M.; Sin, H. S.; Takasu, K.; Chen, J.-S.; Robinson, P. D.; Hua, D. H. Chiral Sulfur-Containing 1,2-Disubstituted Ferrocenes. Tetrahedron, 1998, 54, 7301-7334.
13. Hua, D. H.; Lagneau, N. M.; Chen, Y.; Robben, P. M.; Clapham, G.; Robinson, P. D. Enantioselective Synthesis of Sulfur-Containing 1,2-Disubstituted Ferrocenes. J. Org. Chem. 1996, 61, 4508-4509.
14. Kanato, H.; Tran-Cong, Q.; Hua, D. H. Polarization-Selective Photochromic Reaction of Cyclophane in Glassy Poly(methyl methacrylate) Matrix. Macromolecules 1994, 27, 7907-7913.
15. Hua, D. H.; Dantoing, B.; Robinson, P. D.; Tran-Cong, Q.; Meyers, C. Y. Photoisomerization of tetraethyl 6,8,15,17-tetrahydro-7H,16H,5,18[1′2′]-benzo-9,14-ethenodibenzo[a,h]cyclotetradecene-7,7,16,16-tetracarboxylate. Structure of Tetraethyl 1H,8H-3a,7b[1′,2′]-Benzeno-10a,14b-ethenodibenzo[a,e]dicyclopenta[c,g]-cyclooctene-2,2,9,9(3H,10H)-tetracarboxylate as its Methylene Chloride Solvate. Acta Cryst. 1994, C50, 1090-1093.
16. Hua, D. H. Synthesis of substituted carbon nanotubes. Patent application, 2005, submitted.
17. Park, K. H.; Chhowalla, M.; Iqbal, Z.; Sesti, F. Single-walled carbon nanotubes are a new class of ion channel blockers. J. Biol. Chem. 2003, 278, 50212-50216.
18. Ferrier, M.; De Martino, A.; Kasumov, A.; Gueron, S.; Kociak, M.; Egger, R.; Bouchiat, H. Superconductivity in ropes of carbon nanotubes. Solid State Commun. 2004, 131, 615-623.
19. Baughman, R. H.; Zakhidov, A. A.; de Heer, W. A. Carbon nanotubes—the route toward applications. Science 2002, 297, 787-792.
20. Woolley, A. T.; Cheung, C. L.; Hafner, J. H.; Lieber, C. M. Structural biology with carbon nanotube AFM probes. Chem. Biol. 2000, 7, R193-R204.
21. Dai, H.; Hafner, J. H.; Rinzler, A. G.; Colbert, D. T.; Smalley, R. E. Nanotubes as nanoprobes in scanning probe microscopy. Nature, 1996, 384, 147-149.
22. Harris, P. J. F. Carbon nanotubes and related structures, new materials for the twenty-first century. Cambridge Univ. Press, 1999, New York, N.Y.
23. Chen, J.; Hamon, M. A.; Chen, Y.; Rao, A. M.; Eklund, P. C. Solution properties of single-walled carbon nanotubes. Science, 1998, 282, 95-98.
24. Ito, S.; Wehmeier, M.; Brand, J. D.; Kubel, C.; Epsch, R.; Rabe, J. P.; Mullen, K. Synthesis and self-assembly of functionalized hexa-peri-hexabenzocoronenes. Chem. Eur. J. 2000, 6, 4327.
25. Halleux, A.; Martin, R. H.; King, G. S. D. Syntheses dans la serie des derives polycycliques aromatiques hautement condenses. L′ hexabenzo-1,12,2,3;4,5;6,7;8,9;10,11-coronene, le tetrabenzo-4,5;6,7;11,12;13,14-peropyrene et le tetrabenzo-1,2;3,4;8,9;10,11-bisanthene. Helv. Chim, Acta 1958, 41, 1177-1182.
26. Iyer, V. S.; Wehmeier, M.; Brand, J. D.; Keegstra, M. A.; Mullen, K. Angew. Chem. Int. Ed. Engl. 1997, 36, 1604-1607.
27. Sadhukhan, S. K.; Viala, C.; Gourdon, A. Syntheses of hexabenzocoronene derivatives. Synthesis, 2003, 10, 1521-1525.
28. Isaacs, L.; Haldimann, R. F.; Diederich, F. Tether-directed remote functionalization of buckminsterfullerene: regiospecific hexaadduct formation. Angew. Chem. Int. Ed. Engl. 1994, 33, 2339-2342.
29. Tanguy, G.; Weinberger, B.; des Abbayes, H. Iron pentacarbonyl mediated coupling of benzyl halides to ketones under mild phase transfer conditions. Tetrahedron Lett. 1984, 25, 5529-5532.
30. Barton, J. W.; Shepherd, M. K. Benzocyclo-octenes. Part 5. Thermal rearrangements of benzocyclo-octenes to benzo[a]cyclopropa[c,d]pentalenes. J. Chem. Soc. Perkin Trans. 1, 1986, 961-966.
31. Castellani, M. P.; Wright, J. M.; Geib, S. J.; Rheingold, A. L.; Trogler, W. C. Synthesis, molecular structure, and 2-D NMR analysis of bis(tetraphenylcyclopentadienyl)iron(II). Organomet. 1986, 5, 1116-1122.
32. (a) Brown, A. D. Jr.; Reich, H. Reductive cleavage of ferrocene derivatives. J. Org. Chem. 1970, 35, 1191-1193. (b) Hillman, M.; Austin, J. D.; Kvick, A. Bridged ferrocenes. 12. Metalation and subsequent reactions of ferrocene derivatives with two or three trimethylene bridges. Organomet. 1985, 4, 316-320.
33. Mehr, L.; Becker, E. I.; Spoerri, P. E. Tetraarylcyclopentadienes. J. Am. Chem. Soc. 1955, 77, 984-989.
34. Wirth, T. IBX-new reactions with an old reagent. Angew. Chem. Int. Ed. Engl. 2001, 40, 2812-2814.
35. Hua, D. H.; Miao, S.-W.; Bharathi, S. N.; Katsuhira, T.; Bravo, A. A. Selective Nucleophilic Addition Reactions of Alkyllithium Reagents with N-Trimethylsilyllactams. Synthesis of Cyclic Ketimines. J. Org. Chem. 1990, 55, 3682-3684.
36. Sun, Y.-P.; Fu, K.; Lin, Y.; Huang, W. Functionalized carbon nanotubes: properties and applications. Acc. Chem. Res. 2002, 35, 1096-1104.
37. Chopra, N.; Majumder, M.; Hinds, B. J. Bifunctional carbon nanotubes by sidewall protection. Adv. Funct. Mater. 2005, 15, 858-864.
38. Waybright, S. M.; McAlpine, K.; Laskoski, M.; Smith, M. D.; Bunz, U. H. F. Organometallic dendrimers based on (tetraphenylcyclobutadiene)cyclopentadienylcobalt modules. J. Am. Chem. Soc. 2002, 124, 8661-8666.
39. Cummins, C. H. Synthesis of symmetrical diarylalkynes by double Stille coupling of bis(tributylstannyl)acetylene. Tetrahedron Lett. 1994, 35, 857-860.
40. Ruggli, P.; Lang, F. Azo dyes and intermediates. XIX. Tolan and desoxybenzoin dyes. Helv. Chim. Acta, 1938, 21, 38-50.
41. Harpp, D. N.; Kobayashi, M.; Preparation of thiols using α-trimethylsilyloxy derivatives. Tetrahedron Lett. 1986, 27, 3975-3978.
42. Roubinek, F.; Bydzovsky, V.; Budesinsky, Z. Substituted 5- and 6-quinoxalinecarboxylic acids and their tuberculostatic activity. Coll. Czech. Chem. Commun. 1984, 49, 285-294.
43. Kieczykowski, G. R.; Schlessinger, R. H. Total synthesis of (±)-vernolepin. J. Am. Chem. Soc. 1978, 100, 1938-1940.
44. Abele, E.; Abele, R.; Arsenyan, P.; Lukevics, E. A new pathway for the preparation of diaryl acetylenes. Tetrahedron Lett. 2003, 44, 3911-3913.
45. Comins, D. L.; Brown, J. D. The in situ protection of aldehydes via α-amino alkoxides. Tetrahedron Lett. 1981, 22, 4213-4216.
46. Yokokawa, F.; Asano, T.; Shioiri, T. Total synthesis of the antiviral marine natural product (−)-hennoxazole A. Org. Lett. 2000, 2, 4169-4172.
47. Li, J.-H.; Bigge, C. F.; Williamson, R. M.; Borosky, S. A.; Vartanian, M. G.; Ortwine, D. F. Potent, orally active, competitive N-methyl-D-aspartate (NMDA) receptor antagonists are substrates for a neutral amino acid uptake system in Chinese hamster ovary cells. J. Med. Chem. 1995, 38, 1955-1965.
48. Thompson, A. F. Jr.; Cormwell, N. H. The preparation and properties of aldehydes containing deuterium in the functional group. J. Am. Chem. Soc. 1939, 61, 1374-1376.
49. Mons, M.; Dimicoli, I.; Tardivel, B.; Piuzzi, F.; Robertson, E. G.; Simons, J. P. Energetics of the gas phase hydrates of trans-formanilide: a microscopic approach to the hydration sites of the peptide bond. J. Phys. Chem. A 2001, 105, 969-973.
50. Pedireddi, V. R.; Chatterjee, S.; Ranganathan, A.; Rao, C. N. R. A study of supramolecular hydrogen bonded complexes formed by aliphatic dicarboxylic acids with azaaromatic donors. Tetrahedron, 1998, 54, 9457-9474.
51. Laurence, C.; Berthelot, M.; Evain, K.; Lllien, B The pKHB scales and enthalpies of the hydrogen bond acceptor strength of thioethers, thiols, and disulfides. Can. J. Chem. 2005, 83, 138-145.
52. Beckstein, O.; Tai, K.; Sansom, M. S. P. Not ions alone: barriers to ion permeation in nanopores and channels. J. Am. Chem. Soc. 2004, 126, 14694-14695.
53. Rashin, A. A.; Honig, B. Reevaluation of the Born model of ion hydration. J. Phys. Chem. 1985, 89, 5588-5593.
54. Joseph, S.; Mashl, R. J.; Jakobsson, E.; Aluru, N. R. Electrolytic transport in modified carbon nanotubes. Nano Lett. 2003, 3, 1399-1403.
55. Zhu, F.; Schulten, K. Water and proton conduction through carbon nanotubes as models for biological channels. Biophys. J. 2003, 85, 236-244.
56. Crawford, I.; Maloney, P. C.; Zeitlin, P. L.; Guggino, W. B.; Hyde, S. C.; Turley, H.; Gatter, K. C.; Harris, A.; Higgins, C. F. Immunocytochemical localization of the cystic fibrosis gene product CFTR. Proc. Natl. Acad. Sci. U.S.A. 1991, 88, 9262-9266.
57. Riordan, J. R.; Rommens, J. M.; Kerem, B.; Alon, N.; Rozmahel, R.; Grzelczak, Z.; Zielenski, J.; Lok, S.; Playsic, N.; Chou, J.-L.; Drumm, M. L.; Iannuzzi, M. C.; Collins, F. S.; Tsui, L.-C. Identification of the cystic fibrosis gene: cloning and characterization of complementary DNA. Science, 1989, 245, 1066-1073.
58. Gao, L.; Broughman, J. R.; Iwamoto, T.; Tomich, J. M.; Venglarik, C. J.; Forman, H. J. Synthetic chloride channel restores glutathione secretion in cystic fibrosis airway epithelia. Am. J. Physiol. 2001, 281, L24-L30.
59. Hille, B. in “Ionic Channels of Excitable Membranes”; 2001, 3^{rd} Ed., Sinauer Associates, Sunderland, Mass.
60. Curran, M. E. Potassium ion channels and human disease: phenotypes to drug targets? Curr. Opin. Biotech. 1998, 9, 565-572.
61. Deamer, D. W.; Branton, D. Characterization of nucleic acids by nanopore analysis. Acc. Chem. Res. 2002, 35, 817-825.
62. Thrower, E. C.; Park, H. Y.; So, S. H.; Yoo, S. H.; Ehrlich, B. E. Activation of the inositol 1,4,5-trisphosphate receptor by the calcium storage protein chromogranin A. J. Biol. Chem. 2002, 277, 15801-15806.
63. Andreoli, T. E. Planar lipid bilayer membranes. Methods in Enzym. 1974, 32(Part B), 513-539.
64. Ding, T. T.; Harper, J. D. Analysis of amyloid-β assemblies using tapping mode atomic force microscopy under ambient conditions. In “Amloid, prions, and other protein aggregates”; Wetzel, R. Ed., Methods in Enzymology, 1999, 309, 510-525.
65. Wong, S. S.; Harper, J. D.; Lansbury, P. Y. Jr.; Lieber, C. M. Carbon nanotube tips: high-resolution probes for imaging biological systems. J. Am. Chem. Soc. 1998, 120, 603-604.
66. Selkoe, D. Alzheimer's disease: genes, proteins and therapy. Physiol. Rev. 2001, 81, 741-766.
67. Scheuner, D.; Eckman, C.; Jensen, M.; Song, X.; Citron, M.; Suzuki, N.; Bird, T. D.; Hardy, J.; Hutton, M.; Kukull, W. Secreted amyloid β-protein similar to that in the senile plaques of Alzheimer's disease is increased in vivo by the presenilin 1 and 2 and APP mutations linked to familial Alzheimer's disease. Nature Med. 1996, 2, 864-870.
68. Bitan, G.; Kirkitadze, M. D.; Lomakin, A.; Vollers, S. S.; Benedek, G. B.; Teplow, D. B. Amyloid β-protein (Aβ) assembly: Aβ40 and Aβ42 oligomerize through distinct pathways. Proc. Natl. Acad. Sci. 2003, 100, 330-335.
69. Bitan, G.; Lomakin, A.; Teplow, D. B. Amyloid β-protein oligomerization. J. Biol. Chem. 2001, 276, 35176-35184.
70. Kirkitadze, M. D.; Condron, M. M.; Teplow, D. B. Identification and characterization of key kinetic intermediates in amyloid p-protein fibrillogenesis. J. Mol. Biol. 2001, 312, 1103-1119.
71. Hiura, H.; Ebbesen, T. W.; Tanigaki, K. Opening and purification of carbon nanotubes in high yields. Adv. Mater. 1995, 7, 275-276.
72. Wong, S. S.; Woolley, A. T.; Joselevich, E.; Cheung, C. L.; Lieber, C. M. Covalently functionalized single-walled carbon nanotube probe tips for chemical force microscopy. J. Am. Chem. Soc. 1998, 120, 8557-8558.
73. McKiernan, M.; Huck, J.; Fehrentz, J.-A.; Roumestant, M.-L. Urethane N-carboxyanhydrides from β-amino acids. J. Org. Chem. 2001, 66, 6541-6544.
74. Vieira, E. P.; Hermel, H.; Mohwald, H. Change and stabilization of the amyloid-β(1-40) secondary structure by fluorocompounds. Biochim. Biophys. Acta 2003, 1645, 6-14.
75. Carey, F. A.; Tremper, H. S. Carbonium ion-silane hydride transfer reactions. I. Scope and stereochemistry. J. Am. Chem. Soc. 1968, 90, 2578-2583.
US applications and patents 2004/0265209; 2005/0184294; 2004/0223900; 2003/0089893; U.S. Pat. Nos. 6,841,139; 6,875,412; 6,645,455; 6,835,366; 7,048,999; 2005/0255030; 2005/0006623; 2002/0085968; 2004/0009648; 2005/0025694; 2003/0175199; 2003/0161782; U.S. Pat. No. 6,716,409; 2004/0241077; 2005/0287064; 2002/0112814; U.S. Pat. No. 6,806,397; 2005/0002851; PCT publications WO 2004/007364; WO 02/095098; WO05/121799; WO04/089819; WO99/48810; WO06/012921; WO03/008331; EP application 1605265.

We claim:

1. A method of preparing a synthetic carbon nanotube, comprising:
providing an aryl ferrocene;
ring-closing and carbonylating the aryl ferrocene to form a ferrocenophane;
removing iron and oxidizing the ferrocenophane to form a cyclophane;
oxidizing the cyclophane to form a cyclopentadienone;
condensing the cyclopentadienone with a benzil to form a cyclopentadienone;
Diels-Alder cycloadditioning the cyclopentadienone with a diphenylacetylene to obtain a paracyclophane;
cyclodehydrogenating the paracyclophane to obtain a synthetic carbon nanotube.

2. The method of claim 1, wherein the ferrocenophane is formed from reaction of the aryl ferrocene with Fe(CO)₅.

3. The method of claim 1, wherein the aryl ferrocene contains from one to three cyclopentadiene groups.

4. The method of claim 1, wherein the aryl ferrocene contains one or more functional groups attached to a cyclopentadiene group.

5. The method of claim 1, wherein the diphenylacetylene contains one or more functional groups.

6. The method of claim 5, wherein the one or more functional groups are selected from the group consisting of: halogen, OR, OH, OAc, NR₂, NHAc, SR, O—Si—R₃, and PR₂, wherein the R groups independently may be the same or different and are any desired group including hydrogen; phenyl; substituted phenyl; halogen; C1-C6 alkyl optionally substituted with OR, OH or halogen; diphenyl; and one or more silane-containing protecting groups.

7. The method of claim 1, wherein the diphenylacetylene contains one or more heteroatoms independently in the backbone of one or both phenyl rings.

8. The method of claim 1, wherein the benzil contains one or more protecting groups.

9. The method of claim 8, wherein a protecting group is MOM.

10. A method of preparing a synthetic carbon nanotube comprising:
providing an aryl ferrocene;
forming a cyclopentadienone;
reacting the cyclopentadienone with an optionally substituted diphenylacetylene to form a paracyclophane;
cyclodehydrogenating the paracyclophane to form a synthetic carbon nanotube.

11. The method of claim 10, wherein the cyclopentadienone is formed using a Grubbs' catalyst.

12. An open-ended synthetic carbon nanotube, having a diameter of between 10 Å and 25 Å.

13. The open-ended carbon nanotube of claim 12, having a diameter of 11 Å or larger.

14. The open-ended carbon nanotube of claim 12, having a diameter of greater than 10 Å.

15. The open-ended carbon nanotube of claim 12, having a calcium passing diameter.

16. The open-ended carbon nanotube of claim 12, having a potassium passing diameter.

17. An open-ended synthetic carbon nanotube having a length of 10 Å or greater.

18. An open-ended carbon nanotube having a length less than 10 Å.

19. The open-ended synthetic carbon nanotube of claim 17, having a length between 10 Å and 16 Å.

20. Functionalized open-ended synthetic carbon nanotubes.

21. The functionalized open-ended carbon nanotubes of claim 20, wherein the carbon nanotube comprises one or more heteroatoms in the backbone.

22. The functionalized open-ended carbon nanotubes of claim 21, wherein the carbon nanotube comprises one or more nitrogen atoms in the backbone of one end of the carbon nanotube.

23. The functionalized open-ended carbon nanotubes of claim 20, wherein the carbon nanotube has one or more functional groups independently at one or both ends of the carbon nanotube.

24. The functionalized open-ended carbon nanotubes of claim 23, wherein the functional groups are independently selected from the group consisting of: halogen, amino, thiol, hydroxyl, carboxylic acid, phosphine and metal, including Pt and Pd.

25. The functionalized open-ended carbon nanotubes of claim 20, wherein the carbon nanotube has one or more nitrogen atoms in the backbone at one end of the tube, and one or more hydroxyl groups at the other end of the carbon nanotube.

26. The functionalized open-ended carbon nanotubes of claim 20, wherein the carbon nanotube consists of one or more nitrogen atoms in the backbone of one end of the carbon nanotube and one or more carboxylic acid groups at the other end of the carbon nanotube.

27. The functionalized open-ended carbon nanotubes of claim 20, wherein the carbon nanotube comprises one or more carboxylic acid groups at an end of the carbon nanotube, wherein the carboxylic acid groups are attached to a peptide through an amide bond.

28. The functionalized open-ended carbon nanotubes of claim 20, wherein the carbon nanotube comprises one or more carboxylic acid groups at an end of the carbon nanotube, wherein the carboxylic acid groups are attached to an amino group.

29. The functionalized open-ended carbon nanotubes of claim 28, further comprising a biologically effective compound.

30. The use of a nitrogen-containing synthetic carbon nanotube in the treatment of cystic fibrosis, comprising: administering an effective amount of a composition comprising a nitrogen-containing synthetic carbon nanotube to a patient.
